(19) United States
(12) Patent Application Publication
(10) Pub. No.: US 2010/0160356 A1
(43) Pub. Date: Jun. 24, 2010

(54) NOVEL CYCLOBUTYL COMPOUNDS AS KINASE INHIBITORS

(76) Inventors: Timo Heinrich, Gross-Umstadt (DE); Wolfgang Staehle, Ingelheim (DE); Hartmut Greiner, Weiterstadt (DE); Andree Blaukat, Schriesheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/296,138

(22) PCT Filed: Mar. 8, 2007
(86) PCT No.: PCT/EP2007/001993

§ 371 Date: Oct. 6, 2008

(30) Foreign Application Priority Data
Apr. 7, 2006 (DE) 10 2006 016 426.1

(51) Int. Cl. A61K 31/519; C07D 487/04; A61P 35/00; A61P 9/00; A61P 29/00

(52) U.S. Cl. 514/265.1; 544/280

(57) ABSTRACT

The invention relates to compounds of the formula I, to the preparation and use thereof for the preparation of a medicament for the treatment of diseases, in particular tumours and/or diseases in the formation or course of which protein kinases are involved.

The invention relates to compounds of the formula I

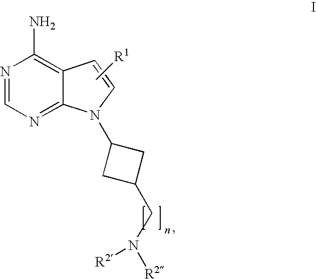

in which
R^{1′} denotes H, Hal, OH, CN, NO₂, NH₂, A, Ar,
R², R²″ each, independently of one another, denote H, A having 1, 2, 3, 4, 5 or 6 C atoms, where R^{2′} and R^{2″} with the N atom to which they are linked, may form a saturated or unsaturated monocyclic heterocycle having no or one further N, O or S atom,
A denotes unbranched, branched or cyclic alkyl having 1, 2, 3, 4, 5, or 6 C atoms, in which one or two CH groups may be replaced by N, furthermore in which one or two CH₂ groups may be replaced by an O, N or S atom and/or by an NH, NA, CONH, Si(CH₃)₂, NHCO, SO₂, —CH═CH— or —C≡C— group and/or, in addition, 1-7H atoms may be replaced by Hal, and in which one or two CH₃ groups may be replaced by NH, NH₂, NAH, NA₂, NHCOOA, NHCONHA, Si(CH₃)₃, CN or Ar,
Ar denotes a mono- or bicyclic aromatic homo- or heterocycle having 1 to 4 N, O and/or S atoms and 5 to 12 skeleton atoms, which may be unsubstituted or mono-, di- or trisubstituted by carbonyl oxygen, Hal, A, OH, OA, NH₂, NHA, NA₂, NO₂, CN, OCN, SCN, COOH, COOA, CONH₂, CONHA, CONA₂, NHCOA, NHCOOA, NHCONH₂, NHSO₂A, CHO, COA, SO₂CH₃ and/or SO₂NH₂,
Hal denotes F, Cl, Br or I and
n denotes 0, 1, 2 or 3,
   and pharmaceutically acceptable salts, derivatives, solvates and stereoisomers thereof, including mixtures thereof in all ratios.

It has been found that the compounds of the formula I are capable of inhibiting, regulating and/or modulating signal transduction mediated by protein kinases. Thus, medicaments and pharmaceutical compositions according to the invention can be effectively employed for the treatment of diseases that are caused, mediated and/or propagated by protein kinases and/or by kinase-mediated signal transduction. Thus, the compounds according to the invention are suitable for the treatment and prophylaxis of angiogenesis, cancer, tumour formation, growth and spread, arteriosclerosis, eye diseases, such as age-induced macular degeneration, choroidal neovascularisation and diabetic retinopathy, inflammatory diseases, arthritis, thrombosis, fibrosis, glomerulonephritis, neurodegeneration, psoriasis, restenosis, wound healing, transplant rejection, metabolic and diseases of the immune system, also autoimmune diseases, cirrhosis, diabetes and diseases of the blood vessels, also instability and permeability and the like in mammals.

Other protein kinase inhibitors are known, for example, from WO 97/28161 or WO 02/92599.

Cancer is a disease whose causes are to be seen, inter alia, in disturbed signal transduction. In particular, deregulated signal transduction via tyrosine kinases plays a central role in the growth and spread of cancer (Blume-Jensen, P. and T. Hunter, Nature 411: 355-365, 2001; Hanahan D. and R. A. Weinberg, Cell 100:57-70, 2000). Tyrosine kinases and in particular receptor tyrosine kinases and the growth factors binding to them may thus be involved in deregulated apoptosis, tissue invasion, metastasis and generally in signal transduction mechanisms which lead to cancer.

As already mentioned, one of the principal mechanisms by which cellular regulation is effected is the transduction of extracellular signals across the membrane that in turn modulate biochemical pathways within the cell. Protein phosphorylation represents one course by which intracellular signals are propagated from molecule to molecule resulting finally in a cellular response. These signal transduction cascades are highly regulated and frequently overlap, as is evident from the existence of many protein kinases as well as phosphatases. Phosphorylation of proteins occurs pre-dominantly at serine, threonine or tyrosine residues, and protein kinases have therefore been classified in accordance with their specificity of the phosphorylation site, i.e. serine/threonine kinases and tyrosine kinases. Since phosphorylation is a very widespread process within cells and since cellular phenotypes are largely influenced by the activity of these pathways, it is currently believed that a large number of conditions and/or diseases are attributable to either aberrant activation or functional mutations in the molecular components of kinase cascades. Consequently, considerable attention has been devoted to the characterisation of these proteins and compounds that are able to modulate their activity (see review article: Weinstein-Oppenheimer et al., Pharma. &. Therap. 88:229-279, 2000). Various possibilities for the inhibition, regulation and modulation of protein kinases encompass, for example, the provision of antibodies, antisense ribozymes and inhibitors. In oncology research, tyrosine kinases, in particular, are highly promising targets. Thus, numerous synthetic small molecules are undergoing clinical development as tyrosine kinase inhibitors for the treatment of cancer, for example Iressa® or Gleevec®. However, numerous problems, such as side effects, dosage, resistance of the tumour, tumour specificity and patient selection, still have to be solved here.

Tyrosine kinases are a class of enzymes which catalyse the transfer of the terminal phosphate of adenosine triphosphate to tyrosine residues in protein substrates. It is thought that tyrosine kinases, through substrate phosphorylation, play an essential role in signal transduction for a number of cellular functions. Although the precise mechanisms of signal transduction are still unclear, it has been shown that tyrosine kinases are important factors in cell proliferation, carcinogenesis and cell differentiation. Tyrosine kinases can be divided into receptor tyrosine kinases and cytosolic tyrosine kinases. Receptor tyrosine kinases have an extracellular part, a transmembrane part and an intracellular part, while cytosolic tyrosine kinases are exclusively intracellular.

Receptor tyrosine kinases consist of a multiplicity of transmembrane receptors with different biological activity. Thus, about 20 different sub-families of receptor tyrosine kinases have been identified. One tyrosine kinase sub-family, known as the EGFR or HER sub-family, consists of EGFR, HER2, HER3 and HER4. Ligands from this sub-family of receptors include epithelial growth factor (EGF), tissue growth factor (TGF-α and β), amphiregulin, HB-EGF, betacellulin and heregulin. Another sub-family of these receptor tyrosine kinases is the insulin sub-family, which includes Ins-R, IGF-IR and IR-R. The PDGF sub-family includes the PDGF-α and -β receptor, CSFIR, c-kit and FLK-II. In addition, there is the FLK family, which consists of the kinase insert domain receptor (KDR) or VEGFR-2, foetal liver kinase-1 (FLK-1), foetal liver kinase-4 (FLK-4) and fms tyrosine kinase-1 (fit-1) or VEGFR-1. The PDGF and FLK family are usually combined in the group of the split kinase domain receptor tyrosine kinases (Laird, A. D. and J. M. Chemington, Expert. Opin. Investig. Drugs 12(1):51-64, 2003) due to the similarities between the two groups. For a detailed discussion of receptor tyrosine kinases, see the paper by Plowman at al., DN & P 7(6):334-339, 1994).

Receptor tyrosine kinases also include TIE2 and its ligands angiopoietin 1 and 2. In the meantime, more and more homologues of these ligands are being found, the action of which has not yet been clarified in detail. A known homologue of TIE2 is TIE1. The TIE receptor tyrosine kinases are expressed selectively on endothelial cells and are involved in processes of angiogenesis and maturing of the blood vessels. They may therefore be a valuable aim, in particular, in diseases of the vascular system and in pathologies in which vessels are utilised or even re-formed. In addition to prevention of neovascularisation and maturing, stimulation of neovascularisation may also be a valuable aim for active ingredients.

A further receptor tyrosine kinase is MET. Its role in human oncogenesis, and the possibility of inhibition of HGF (hepatocyte growth factor) dependent Met activation is described by S. Berthou et al. (Oncogene 23(31): 5387-5393, 2004).

Cytosolic tyrosine kinases likewise consist of a multiplicity of sub-families, including SRC, FRK, BTK, CSK, ABL, ZAP70, FES/FPS, FAK, JAK, ACK and LIMK. Each of these sub-families is further divided into various sub-groups. Thus, for example, the Src sub-family represents one of the largest sub-families. It includes SRC, YES, FYN, LYN, LCK, BLK, HCK, FGR and YRK. The SRC enzyme sub-family has been linked to oncogenesis. For a more detailed discussion of cytosolic tyrosine kinases, see the paper by Bolen, Oncogene, 8:2025-2031, 1993.

Both receptor tyrosine kinases and cytosolic tyrosine kinases are involved in cellular signal transfer pathways leading to the above-mentioned conditions, such as cancer, psoriasis and hyperimmune reactions.

The other large group of protein kinases, the serine/threonine kinases, includes, for example, the checkpoint kinases CHK1 and CHK2 and SGK (human serum- and glucocorticoid-dependent kinase), of which the sub-types SGK-1, -2 and -3 are known.

The invention now had the object of finding novel compounds having advantageous therapeutic properties which can be used for the preparation of medicaments.

Thus, the identification and provision of chemical compounds which inhibit, regulate and/or modulate signal transduction of various kinases is desirable and was therefore an aim of the present invention.

The compounds of the formula I according to the invention are now distinguished, surprisingly, by three properties:

1. They are capable of inhibiting a multiplicity of protein kinases instead of acting specifically on only a particular kinase. This spectrum activity is of major importance in the treatment of tumour diseases since tumour cells typically deviate to alternative signal transduction cascades during proliferation if a certain cascade is blocked.

2. They do not inhibit InsR or only do so to a small extent. Substances which inhibit IGF1R often also inhibit InsR since these two receptors are structurally related to one another. However, comparably strong inhibition of InsR is generally undesired since this may result in side effects (for example development of diabetes).

The compound trans-5-(3-benzyloxyphenyl)-7-(3-pyrrolidin-1-ylmethyl-cyclobutyl)-7H-pyrrolo[2,3-d]pyrimidin-4-ylamine known from WO 02/092599 (Ex. 64), which was later shown to be particularly effective under the code name NVP-ADW742 (Mitsiades et al., Cancer Cell 5:221-230, 2004), has, for example, a selectivity for IGF1R which is only about 5 times higher compared with InsR (IC₅₀ (ELISA) IGFR=0.165 μM, InsR=0.76), than the compounds according to the invention, where this ratio is approximately 10.

3. Even as free bases, they have unexpectedly high solubility in aqueous buffer systems compared with compounds of the prior art. High solubility under physiological conditions and consequently high bioavailability are of major importance for a medicament active ingredient.

Important types of cancer which can be treated using a compound according to the invention include breast cancer, prostate cancer, colorectal cancer, brain cancer, small-cell lung cancer, non-small-cell lung cancer, multiple myeloma, renal cell carcinoma, endometrial carcinoma, squamous epithelial cancer, bladder cancer, stomach cancer, pancreatic cancer, liver cancer, chronic leukaemia and acute leukaemia.

The present invention thus relates to the use of the compounds of the formula I for the prevention and/or treatment of diseases in connection with unregulated or disturbed kinase activity.

In addition, the compounds according to the invention can be used to achieve additive or synergistic effects in certain existing cancer chemotherapies and radiotherapies and/or to restore the efficacy of certain existing cancer chemotherapies and radiotherapies.

### DESCRIPTION OF THE INVENTION

It has been found that the compounds of the formula I and salts thereof have very valuable pharmacological properties while being well tolerated. In particular, it has been found that the compounds of the formula I according to the invention having good solubility are effective kinase inhibitors, exhibiting a pronounced spectrum activity.

In general, all radicals which occur more than once may be identical or different, i.e. are independent of one another. Above and below, the radicals and parameters have the meanings indicated for the formula I, unless expressly indicated otherwise.

Accordingly, the invention relates, in particular, to the compounds of the formula I in which at least one of the said radicals has one of the preferred meanings indicated below.

Hal denotes fluorine, chlorine, bromine or iodine, in particular fluorine or chlorine.

A denotes alkyl, is unbranched (linear), branched or cyclic, has 1, 2, 3, 4, 5 or 6 C atoms and may be substituted by Ar.

Thus, A denotes, for example, methyl, furthermore ethyl, propyl, isopropyl, butyl, isobutyl, sec-butyl or tent-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl.

A furthermore preferably denotes alkyl, in which one or two CH groups may be replaced by N, furthermore in which one or two CH₂ groups may be replaced by O, N or S atoms and/or by NH, NA, SO₂, CONH, Si(CH₃)₂, NHCO, —C≡C— or —CH═CH— groups and/or, in addition, 1-7H atoms may be replaced by F and/or Cl, such as, for example, methyl, ethyl, propyl, isopropyl, butyl, isobutyl, sec-butyl, tert-butyl, pentyl, hexyl, trifluoromethyl, pentafluoroethyl, 1,1-difluoromethyl, 1,1,1-trifluoroethyl, methoxy, ethoxy, n-propoxy, isopropoxy, n-butoxy, isobutoxy, sec-butoxy or tert-butoxy, and in which one or two CH₃ groups may be replaced by OH, NH₂, NAH, Si(CH₃)₃, NA₂ or CN.

Cycloalkyl preferably denotes cyclopropyl, cyclobutyl, cyclopentyl, cyclohexyl or cycloheptyl.

Ar denotes, for example, unsubstituted phenyl, naphthyl or biphenyl, furthermore preferably, for example, phenyl, naphthyl or biphenyl, each of which is mono-, di- or trisubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamide, methylsulfonamido, ethylsulfonamido, propylsulfonamido, butylsulfonamido, dimethylsulfonamido, phenylsulfonamido, carboxyl, methoxycarbonyl, ethoxycarbonyl, aminocarbonyl.

Ar furthermore denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)-phenyl, o-, m- or p-(N-ethylamino)phenyl, o-, m- or p-(N,N-diethylamino)-phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl, (4-methoxyphenyl)methyl, (3-methoxyphenyl)methyl, (4-methoxyphenyl)ethyl, (3-methoxyphenyl)ethyl.

Ar furthermore preferably denotes 2-, 3- or 4-phenyl, 2-, 3- or 4-phenylmethyl, 2-, 3- or 4-phenylethyl, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 3- or 4-pyridylmethyl, 2-, 3- or 4-pyridylethyl, 2-, 4-, 5- or 6-pyrimidinyl, 2-, 3-, 5-, or 6-pyrazin-1- or 4-yl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, 1-, 2-, 3-, 4-, 5-, 6- or 7-indolyl, 2-, 3-, 4- or 5-isoindolyl, 2-, 3-, 4-, 5- or 6-indazolyl, 3-, 4-, 5- or 6-benzotriazolyl, 2-, 6, - or 8-purinyl, 1-, 2-, 4- or 5-benzimidazolyl, 1-, 3-, 4-, 5-, 6- or 7-benzopyrazolyl, 2-, 4-, 5-, 6- or 7-benzoxazolyl, 4-, 5-, 6- or 7-benzoxazolyl-2-one, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 3-, 4-, 5-, 6-, 7- or 8-isoquinolinyl, 3-, 4-, 5-, 6-, 7- or 8-quinolinyl, 2-, 4-, 5-, 6-, 7- or 8-quinazolinyl, 5- or 6-quinoxalinyl, 4-, 5-, or 6-phthalazinyl, 2-, 3-, 5-, 6-, 7- or 8-2H-benzo-1,4′-oxazinyl, further preferably 1,3-benzodioxol-5-yl, 1,4-benzodioxan-6-yl, 2,1,3-benzothiadiazol-4- or -5-yl, 2,1,3-benzoxadiazol-5-yl or benzofuran-4, -5, -6, or -7-yl, each of which is unsubstituted or mono-, di- or trisubstituted, for example, by carbonyl oxygen, F, Cl, Br, methyl, ethyl, propyl, phenyl, benzyl, —CH₂-cyclohexyl, hydroxyl, methoxy, ethoxy, amino, methylamino, dimethylamino, nitro, cyano, carboxyl, methoxycarbonyl, aminocarbonyl, methylaminocarbonyl, dimethylaminocarbonyl, acetamino, ureido, methylsulfonylamino, formyl, acetyl, aminosulfonyl and/or methylsulfonyl.

The heterocyclic radicals may also be partially or fully hydrogenated and also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 2-, 3-, 5- or 6-piperidin-1 or 4-yl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydrobenzo-1,4-dioxin-4-, -5 or -6-yl or 2,3-dihydro-2-oxofuranyl,

The term “substituted” preferably relates to the substitution by the above-mentioned substituents, where a plurality of different degrees of substitution are possible, unless indicated otherwise.

All physiologically acceptable salts, derivatives, solvates and stereoisomers of these compounds, including mixtures thereof in all ratios, are also in accordance with the invention.

The compounds of the formula I may have one or more centres of chirality. They may accordingly occur in various enantiomeric forms and exist in racemic or optically active form. The invention therefore also relates to the optically active forms (stereoisomers), the enantiomers, the racemates, the diastereomers and hydrates and solvates of these compounds.

Since the pharmaceutical efficacy of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

An elegant method for the resolution of racemates containing ester groups (for example acetyl esters is the use of enzymes, in particular esterases.

A preferred group of compounds of the formula I conforms to the formula II

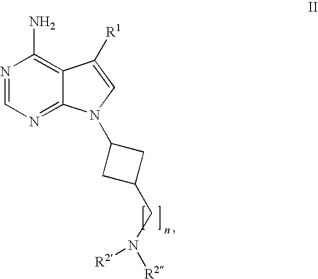

in which R¹, R² and n have the meaning indicated for the formula I and pharmaceutically acceptable salts, derivatives, solvates and stereoisomers thereof, including mixtures thereof in all ratios.

Further preferred sub-groups of compounds of the formulae I and II can be expressed by the following sub-formulae A to E, which conform to the formulae I or II, but in which

in sub-formula A
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
and R² and n have the meaning indicated for the formula I,
   in sub-formula B
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
R^{2′}, R^{2″} are H or together form a butylene unit and
n denotes 1,
   in sub-formula C
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
R^{2′}, R^{2″} are each, independently of one another, H or unbranched or branched alkyl having 1, 2, 3 or 4 C atoms, where R^{2′} and R^{2″} together form an ethylene, propylene, butylene or pentylene unit and
n denotes 1,
   in sub-formula D
R¹ denotes propan-1-olyl, propen-1-olyl, propyn-1-olyl, or phenyl, indolyl, indazolyl, benzofuranyl, benzotriazolyl, benzimidazolyl-2-one, benzoxazolyl-2-one, furyl, thienyl, thiazolyl, pyridyl or pyrimidinyl, each of which is unsubstituted or mono- or disubstituted by hydroxyl, amino, fluorine, butoxy, acetamido, t-butoxycarbonylamino, nitro, benzyl, (dimethylphenylsilanyl)methoxy, dimethylphenylsilanyloxy, methanesulfonyl, sulfonamido, methanesulfonamido, methyl, 2-propyl, trifluoromethyl, trifluoromethoxy, trifluoromethanesulfonic acid, benzylamino, N-benzylpropane-1,3-diamino,
and R² and n have the meaning indicated for the formula I,
   in sub-formula E
R¹ denotes propan-1-olyl, propen-1-olyl, propyn-1-olyl, or phenyl, indolyl, indazolyl, benzofuranyl, benzotriazolyl, benzimidazolyl-2-one, benzoxazolyl-2-one, furyl, thienyl, thiazolyl, pyridyl or pyrimidinyl, each of which is unsubstituted or mono- or disubstituted by hydroxyl, amino, fluorine, butoxy, acetamido, t-butoxycarbonylamino, nitro, benzyl, (dimethylphenylsilanyl)methoxy, dimethylphenylsilanyloxy, methanesulfonyl, sulfonamido, methanesulfonamido, methyl, 2-propyl, trifluoromethyl, trifluoromethoxy, trifluoromethanesulfonic acid, benzylamino, N-benzylpropane-1,3-diamino,
R², R²″ are H or together form a butylene unit and
n denotes 1
   and pharmaceutically acceptable salts, derivatives, solvates and stereo-isomers thereof, including mixtures thereof in all ratios.

Particular preference is given to sub-formulae A to E of the compounds of the formula II. Very particular preference is given to compounds selected from the compounds shown in Table 1 and pharmaceutically acceptable salts, derivatives, solvates and stereoisomers thereof, including mixtures thereof in all ratios.

The melting points, solubility values and IC50 values indicated in Table 1 relate, if no anion is indicated, to the free base which is in the trans-configuration. If a compound cannot be obtained in crystalline form, the material nature at room temperature is indicated. The solubility data relate to the conditions indicated in Example C. The 1050 values indicated were obtained by the flashplate method (see Examples B), unless indicated otherwise (ELISA, see Examples B).

TABLE 1

| | | | Solubility | Melting point |
|---|---|---|---|---|
| | | IC50 (μM) | [μg/ml] | [° C.] |
| 1 | 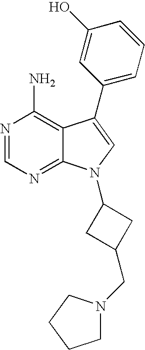 | IGF = 0.060 (2.4 ELISA) InsR = 2.1 (ELISA) FAK = 1.8 (0.077 ELISA) SGK = 0.012 TGF = 0.120 TIE = 0.520 VEGF = 0.180 PDGF = 0.097 | 1071 | 193-194 |
| 2 | 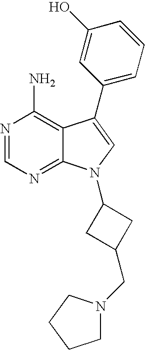 | IGF = 0.11 (5.9 ELISA) FAK = 2.7 (0.26 ELISA) SGK = 7.4 TGF = 0.14 TIE = 0.87 VEGF = 0.195 PDGF = 0.12 | 1147 | (bistosylate) |
| 3 | 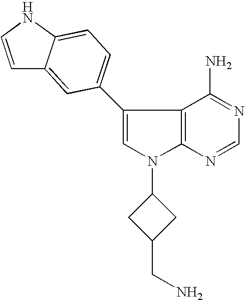 | IIGF = 0.93; (5 ELISA) InsR = (12 ELISA) FAK = 7.9; (0.37 ELISA) TGF = 0.18 TIE = 0.38 VEGF = 0.01 PDGF = 0.15 PDK = 6.1 | 1326 | 142-144 |
| 4 | 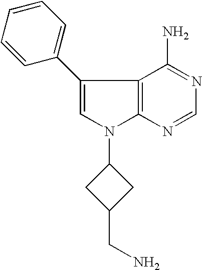 | IGF = 7.4 FAK = 1.1 (ELISA) TGF = 2.8 TIE = 7.4 VEGF = 1.1 PDGF = 0.92 | | 84-86 |
| 5 | 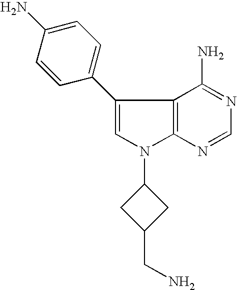 | IGF = 2.3 FAK = SGK = TGF = 2.9 TIE = VEGF = 0.630 PDGF = 0.630 | 1090 | 319.5-321 (decomposition) |
| 6 | 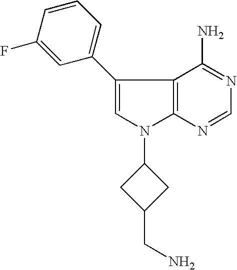 | IGF = 7.5 FAK = 1.4 (ELISA) TGF = 1.7 VEGF = 1.8 PDGF = 0.7 | | 101-102 |
| 7 | 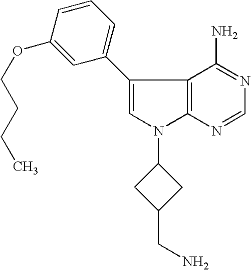 | IGF = 1.2 TIE = 0.91 VEGF = 9.1 | 1122 | (HCl) |
| 8 | 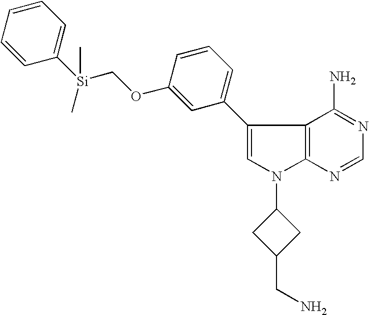 | IGF = 0.32 (0.81 ELISA) InsR = 9.5 (ELISA) FAK = 16 (ELISA) Met = 1.1 | | 172-174.5 (TFA) |
| 9 | 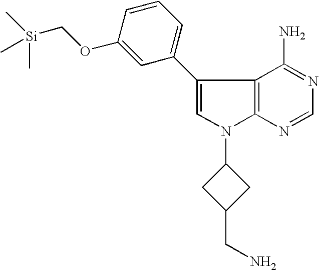 | IGF = 0.13 (0.57 ELISA) InsR = 5.5 (ELISA) FAK = 14 (ELISA) | | 83-84 (TFA) |
| 10 | 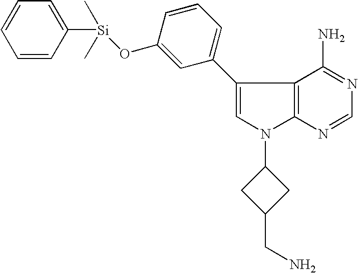 | | | |
| 11 | 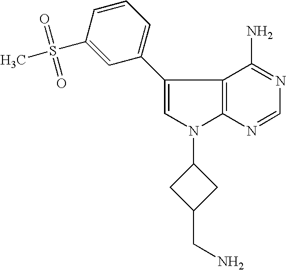 | IGF = 10 (ELISA) FAK = 28 (ELISA) | >1000 | |
| 12 | 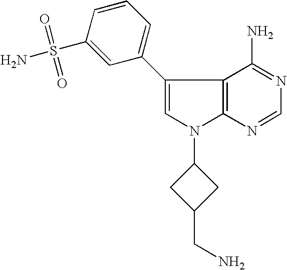 | | | |
| 13 | 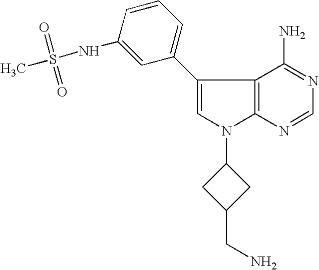 | FAK = 28 (ELISA) VEGF = 6.5 PDGF = 5.8 | | 82-84 |
| 14 | 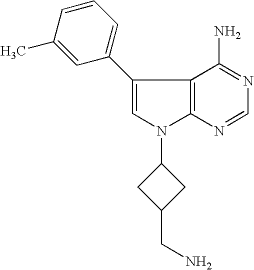 | | | |
| 15 | 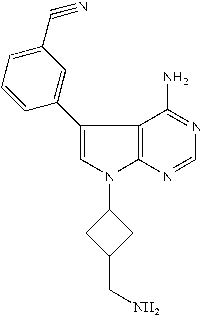 | | | 186.5-189 (TFA) |
| 16 | 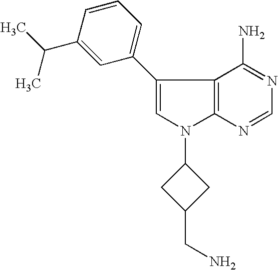 | IGF = (22 ELISA) FAK = (28.5 ELISA) TGF = 1.5 | | 93-94.5 |
| 17 | 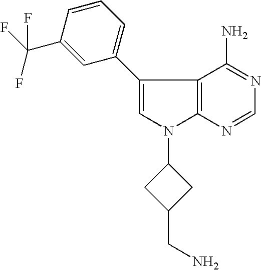 | FAK = (25 ELISA) TGF = 2.7 | | (HCl) |
| 18 | 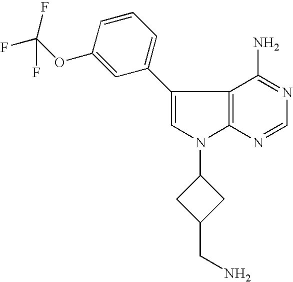 | IGF = (22 ELISA) FAK = (17.5 ELISA) TGF = 1 | | 88-90 |
| 19 | 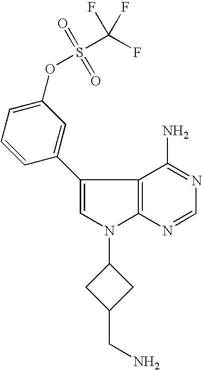 | | | |
| 20 | 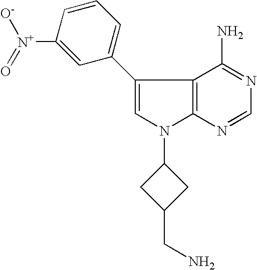 | FAK = (4 ELISA) TGF = 0.7 | | 53-55 |
| 21 | 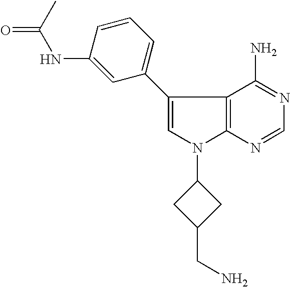 | FAK = (17 ELISA) TGF = 6.5 | | Oil |
| 22 | 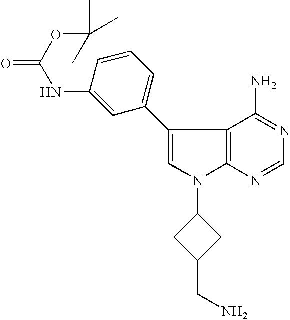 | | | |
| 23 | 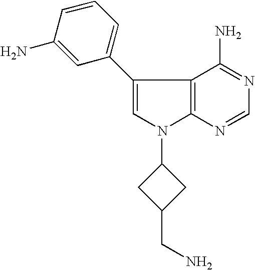 | IGF = 7.6 (14 ELISA) FAK = (1.21 ELISA) SGK = 1.95 TGF = 0.81 | | Oil |
| 24 | 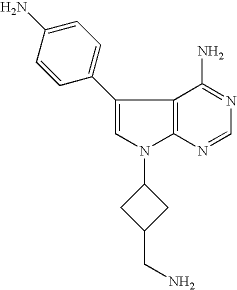 | IGF = 2.3 (9.2 ELISA) FAK = 3.3 (ELISA) TGF = ..9 TIE = 1.5 VEGF = 0.63 PDGF = 0.63 | 1090 | 319.5-321 (decomposition) |
| 25 | 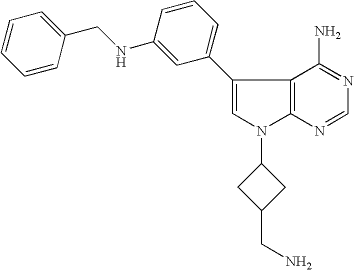 | IGF = 0.82 (3 ELISA) SGK = 3.6 TGF = 3.7 TIE = 1.5 VEGF = 0.39 PDGF = 1.2 | | 204-206 |
| 26 | 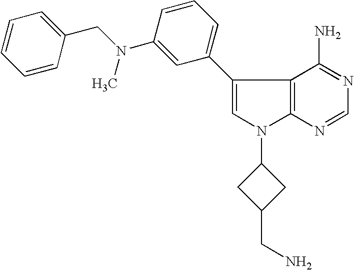 | IGF = (11 ELISA) TGF = 1.1 VEGF = 2.7 | | 197-200 |
| 27 | 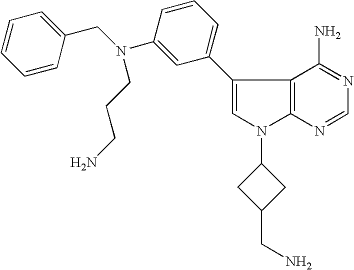 | | | |
| 28 | 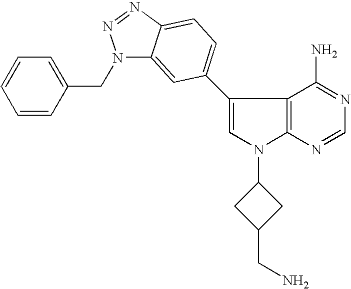 | | | |
| 29 | 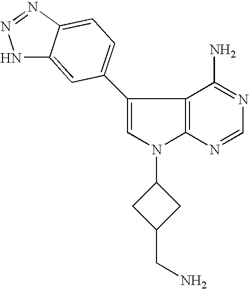 | | | |
| 30 | 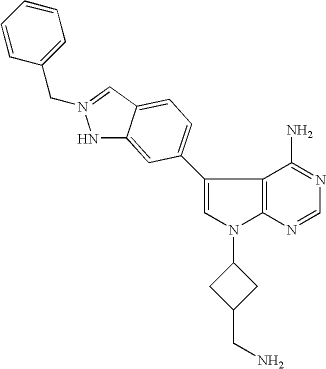 | | | |
| 31 | 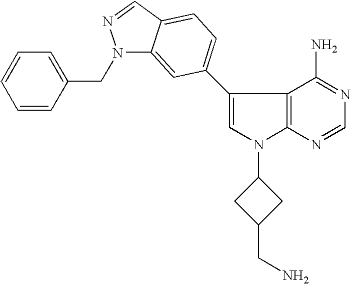 | | | |
| 32 | 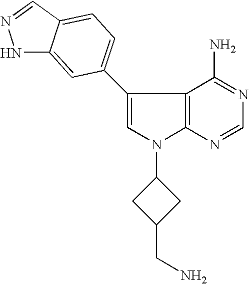 | | | |
| 33 | 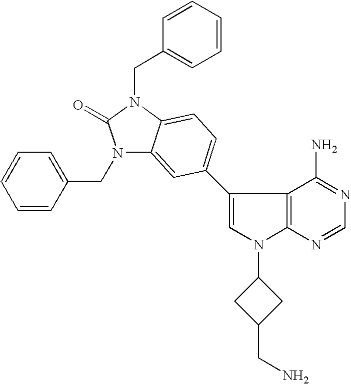 | IGF = (20 ELISA) FAK = (27 ELISA) TIE = 0.37 PDGF = 6.85 | | 72.5-74 |
| 34 | 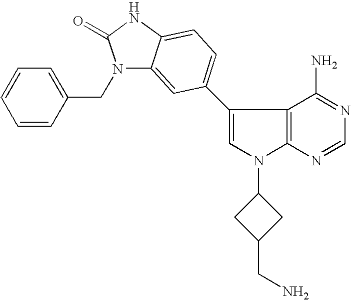 | | | |
| 35 | 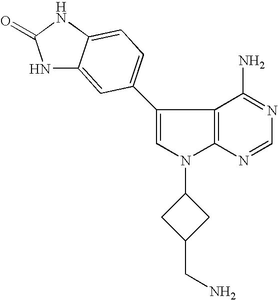 | | | |
| 36 | 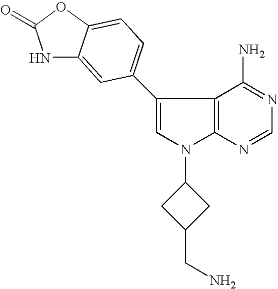 | | | |
| 37 | 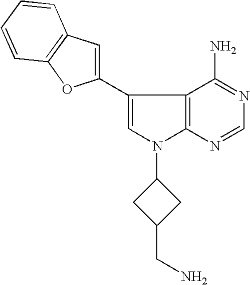 | IGF = 8.4 (29 ELISA) FAK = (6.1 ELISA) SGK = 6.1 TGF = 0.21 TIE = 1.1 VEGF = 0.16 PDGF = 0.17 PDK = 6.2 | | 202.5-204.5 (TFA) |
| 38 | 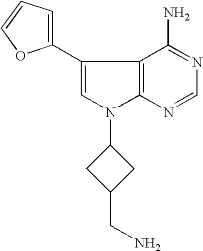 | IGF = (17 ELISA) FAK = (18 ELISA) VEGF = 2.3 SGK = 2.6 CHK = 8.8 | | 207.5-209 (TFA) |
| 39 | 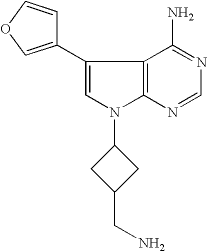 | FAK = (15 ELISA) SGK = 2.5 TGF = 8.5 | | Oil (TFA) |
| 40 | 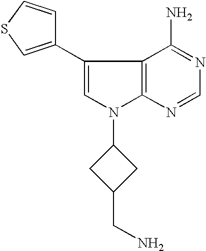 | IGF = 18 (ELISA) FAK = 2.6 (ELISA) SGK = 8.4 TGF = 2.2 TIE = 9.3 VEGF = 1.8 PDGF = 0.75 | | 109-111 |
| 41 | 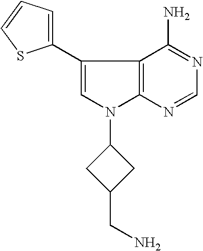 | IGF = 5.6; (6.2 ELISA) FAK = (3.8 ELISA) SGK = 1.6 TGF = 2.7 TIE = 4.8 VEGF = 1.2 PDK = 5.4 | | 206-207.5 (TFA) |
| 42 | 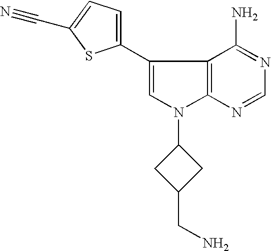 | | | Oil (TFA) |
| 43 | 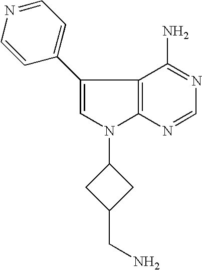 | IGF = (27 ELISA) TGF = 0.15 | | >195 (decomposition) (TFA) |
| 44 | 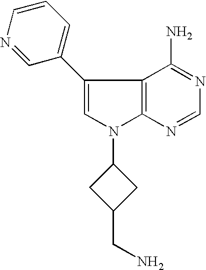 | IGF = 13 (ELISA) TGF = 0.95 | | (TFA) |
| 45 | 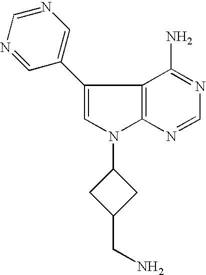 | | | |
| 46 | 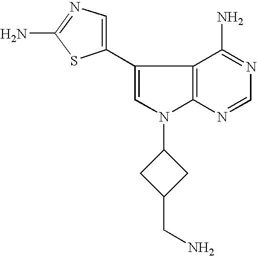 | | | |
| 47 | 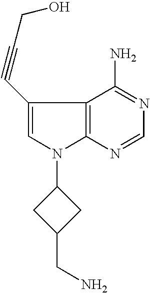 | SGK = 2.55 | | (TFA) |
| 48 | 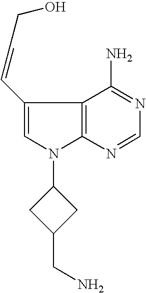 | | | |
| 49 | 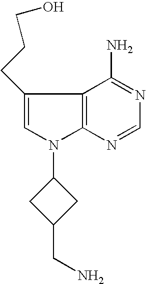 | | | |
| 50 | 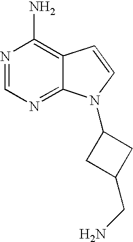 | | | Oil (TFA) |
| 51 | 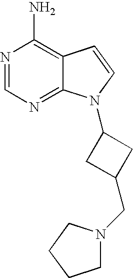 | | | Oil |
| 52 | 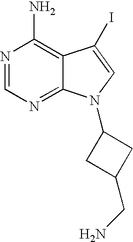 | IGF = (7.5 ELISA) SGK = 0.25 TIE = 2.6 VEGF = 0.715 PDGF = 1.1 | | 241-242.5 (HCl) |
| 53 | 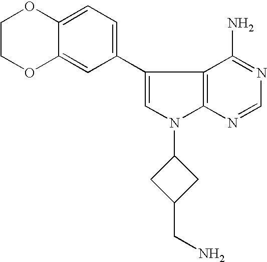 | IGF = (5.3 ELISA) FAK = (1.9 ELISA) TGF = 1.6 TIE = 2 VEGF = 1.4 PDGF = 1.1 | | 324-325.5 |
| 54 | 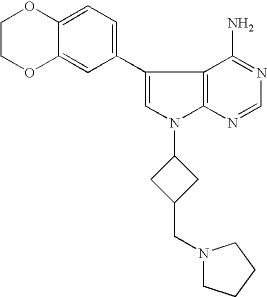 | FAK = (28 ELISA) | | Oil |
| 55 | 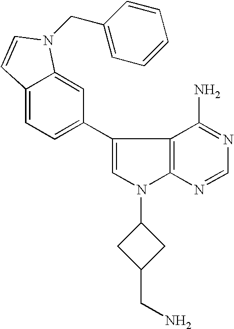 | IGF = 1.6 (3.5 ELISA) FAK = (24 ELISA) TIE = 0.68 VEGF = 0.19 | | Oil |
| 56 | 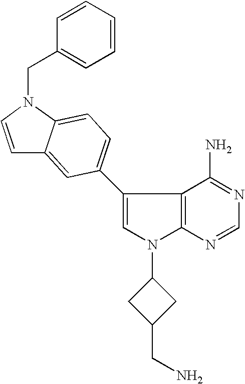 | IGF = 1.4 (4.1 ELISA) TGF = 5 TIE = 0.495 VEGF = 0.061 | | Oil |
| 57 | 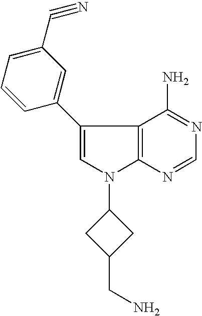 | FAK = (25 ELISA) TGF = 6.1 | | 186.5-189 (TFA |
| 58 | 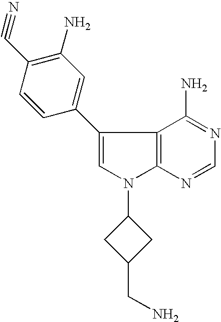 | FAK = (1.1 ELISA) TGF = 1.1 VEGF = 0.61 PDGF = 0.43 | | decomposition at 220 |
| 59 | 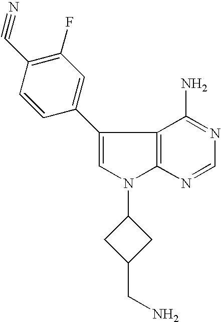 | FAK = (4.1 ELISA) TGF = 8.3 PDGF = 0.62 | | Oil |
| 60 | 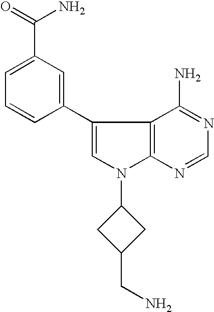 | TGF = 0.53 | | Oil (TFA) |
| 61 | 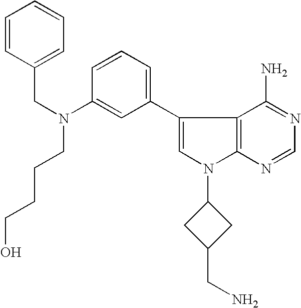 | Tie = 3.9 | | Oil (TFA) |
| 62 | 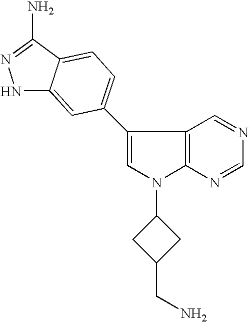 | | | Oil (TFA) |
| 63 | 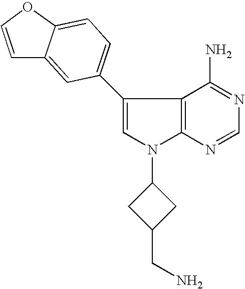 | FAK = (0.63 ELISA) TGF = 0.055 TIE = 1.8 VEGF = 0.29 PDGF = 0.31 PDK = 5.7 | | 156-159 |
| 64 | 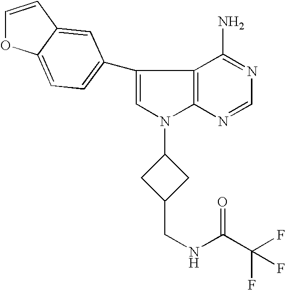 | FAK = (3.6 ELISA) SGK = 4.4 TGF = 0.33 TIE = 2.4 VEGF = 1.06 PDGF = 2.45 | | 92-93.5 |
| 65 | 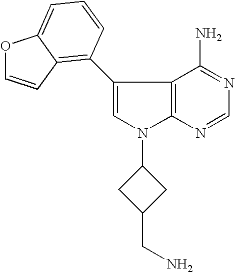 | FAK = (0.42 ELISA) TGF = 0.045 TIE = 2.9 VEGF = 1.325 PDGF = 0.94 | | |
| 66 | 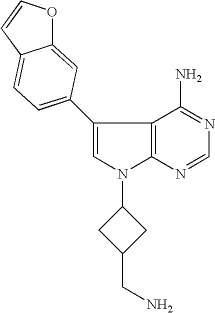 | | | |
| 67 | 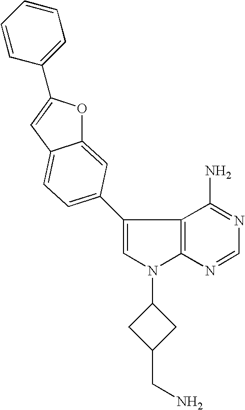 | | | |
| 68 | 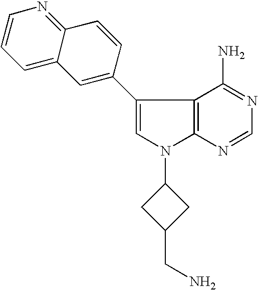 | | | Oil |
| 69 | 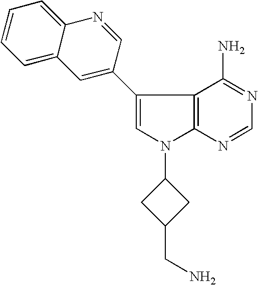 | IGF = (25 ELISA) FAK = (8 ELISA) TGF = 0.039 | | Oil |
| 70 | 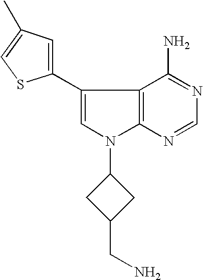 | IGF = (8.5 ELISA) FAK = (7.1 ELISA) SGK = 4.45 TGF = 1.4 TIE = 2.6 PDGF = 2.5 | | Oil (TFA) |
| 71 | 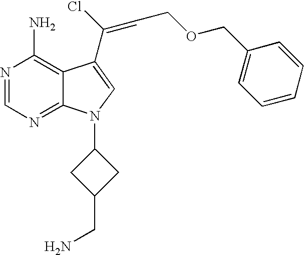 | IGF = (21 ELISA) TGF = 5.5 | | Oil |

Pharmaceutically or physiologically acceptable derivatives are taken to mean, for example, salts of the compounds according to the invention and also so-called prodrug compounds. Such derivatives are known in the person skilled in the art. A review of physiologically tolerated derivatives is given in Burger's Medicinal Chemistry And Drug Discovery, 5th Edition, Vol 1: Principles and Practice. Prodrug compounds are taken to mean compounds of the formula I which have been modified with, for example, alkyl or acyl groups, sugars or oligopeptides and which are rapidly cleaved or liberated in the organism to give the effective compounds according to the invention. These also include biodegradable polymer derivatives of the compounds according to the invention, as described, for example, in Int. J. Pharm. 115:61-67, 1995.

Suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methyl-sulfonates or p-toluenesulfonates.

Solvates of the compounds of the formula I are taken to mean adductions of inert solvent molecules onto the compounds of the formula I which form owing to their mutual attractive force. Solvates are, for example, hydrates, such as monohydrates or dihydrates, or alcoholates, i.e. addition compounds with alcohols, such as, for example, with methanol or ethanol.

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence: improved treatment, healing, prevention or elimination of a disease, syndrome, condition, complaint, disorder or prevention of side effects or also reduction in the progress of a disease, condition or disorder. The term “therapeutically effective amount” also encompasses the amounts which are effective for increasing normal physiological function.

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds.

The present invention furthermore relates to a process for the preparation of compounds of the formula I and physiologically acceptable salts, derivatives, solvates and stereoisomers thereof, characterised in that, in a first step, a compound of the formula VI

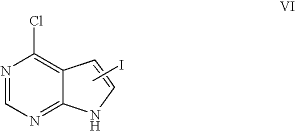

is condensed with a compound of the formula V

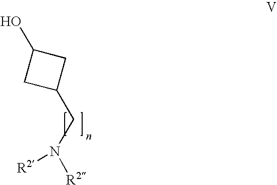

to give a compound of the formula IV

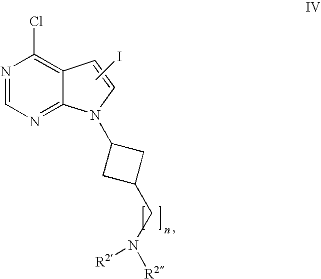

which is further linked to a desired radical R¹ to give a compound of the formula III

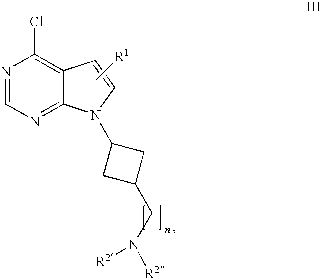

which is finally reacted with NH₃ to give a compound of the formula I.

Finally, a base or acid of the formula I can be converted into one of its salts.

The starting materials for the process according to the invention are generally known. If they are novel, they can be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York).

The compounds of the formula I and also the starting materials for their preparation are prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions as are known and suitable for the said reactions. Use can also be made here of variants known per se which are not mentioned here in greater detail.

The reactions described above are generally carried out in an inert solvent. Suitable inert solvents for the reactions described above are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, N-methylpyrrolidone (NMP), dimethylacetamide or dimethylformamide (DMF); nitrites, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents. Preference is given to sulfoxides, such as dimethyl sulfoxide (DMSO).

The amount of solvent is not crucial, 5 g to 500 g of solvent can preferably be added per g of the product to be formed.

In general, the process is carried out at a pressure of 1 to 200 bar, but preferably at atmospheric pressure.

Depending on the conditions used, the reaction temperature for the reactions described above is between about −10 and 200° C., normally between −5 and 100° C., preferably between 0 and 80° C.

Depending on the conditions used, the reaction time is between a few minutes and a number of days, preferably in the region of a number of hours.

The reaction can also be carried out in the heterogeneous phase, in which case use is preferably made of an aqueous phase and a benzene or toluene phase. Use is made here of a phase-transfer catalyst, such as, for example, tetrabutylammonium iodide, and optionally an acylation catalyst, such as, for example, dimethylaminopyridine.

A base of the formula I obtained can be converted into the associated acid-addition salt using an acid. Suitable for this reaction are acids which give physiologically acceptable salts. Thus, it is possible to use inorganic acids, for example sulfuric acid, hydrohalic acids, such as hydrochloric acid or hydrobromic acid, phosphoric acids, such as orthophosphoric acid, nitric acid, sulfamic acid, furthermore organic acids, in detail aliphatic, alicyclic, araliphatic, aromatic or heterocyclic mono- or polybasic carboxylic, sulfonic or sulfuric acids, such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethanesulfonic acid, ethanedisulfonic acid, 2-hydroxyethanesulfonic acid; benzenesulfonic acid, p-toluenesulfonic acid, naphthalenemono- and -disulfonic acids, laurylsulfuric acid.

If desired, the free bases of the formula I can be liberated from their salts by treatment with strong bases, such as sodium hydroxide, potassium hydroxide, sodium carbonate or potassium carbonate, so long as no other acidic groups are present in the molecule.

Compounds of the formula I can furthermore be obtained by liberating them from one of their functional derivatives by treatment with a solvolysing or hydrogenolysing agent.

Preferred starting materials for the solvolysis or hydrogenolysis are those which otherwise conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ denotes an amino-protecting group, instead of an HN group and/or those which carry a hydroxyl-protecting group instead of the H atom of a hydroxyl group, for example those which conform to the formula I, but carry a —COOR″ group, in which R″ denotes a hydroxyl-protecting group, instead of a —COON group.

Preferred starting materials are also the oxadiazole derivatives, which can be converted into the corresponding amidino compounds.

It is also possible for a plurality of—identical or different—protected amino and/or hydroxyl groups to be present in the molecule of the starting material. If the protecting groups present are different from one another, they can in many cases be cleaved off selectively.

The expression “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, C atoms. The expression “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl, butyryl; aralkanoyl, such as phenylacetyl; aroyl, such as benzoyl or tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl), 2-iodoethoxycarbonyl; aralkoxycarbonyl, such as CBZ (“carbobenzoxy”), 4-methoxybenzyloxycarbonyl, FMOC; arylsulfonyl, such as Mtr. Preferred amino-protecting groups are BOC and Mtr, furthermore CBZ, Fmoc, benzyl and acetyl.

Furthermore, free amino groups can be acylated in a conventional manner using an acid chloride or anhydride or alkylated using an unsubstituted or substituted alkyl halide, or reacted with CH₃—C(═NH)—OEt, advantageously in an inert solvent, such as dichloromethane or THF, and/or in the presence of a base, such as triethylamine or pyridine, at temperatures between −60 and +30° C.

The expression “hydroxyl-protecting group” is likewise known in general terms and relates to groups which are suitable for protecting a hydroxyl group against chemical reactions, but which are easy to remove after the desired chemical reaction has been carried out elsewhere in the molecule. Typical of such groups are the above-mentioned unsubstituted or substituted aryl, aralkyl or acyl groups, furthermore also alkyl or silyl groups. The nature and size of the hydroxyl-protecting groups is not crucial since they are removed again after the desired chemical reaction or reaction sequence; preference is given to groups having 1-20, in particular 1-10, C atoms. Examples of hydroxyl-protecting groups are, inter glia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature, RT).

The BOC, OBut and Mtr groups can, for example, preferably be cleaved off using TFA in dichloromethane or using approximately 3 to 5N HCl in dioxane at 15-30° C., the FMOC group can be cleaved off using an approximately 5 to 50% solution of dimethylamine, diethylamine or piperidine in DMF at 15-30° C.

Hydrogenolytically removable protecting groups (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° C. and pressures between about 1 and 200 bar, preferably at 20-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 20-30° C.

Esters can be saponified, for example, using acetic acid or using NaOH or KOH in water, water/THF or water/dioxane, at temperatures between 0 and 100° C.

Further methods for the removal of protecting groups is described, for example, in Theodora W. Green, Peter G. M. Wuts: Protective Groups in Organic Synthesis, 3rd Edition John Wiley & Sons (1999).

Compounds of the formula I according to the invention may be chiral owing to their molecular structure and accordingly occur in various enantiomeric forms. They can therefore exist in racemic or in optically active form.

Since the pharmaceutical efficacy of the racemates or stereoisomers of the compounds according to the invention may differ, it may be desirable to use the enantiomers. In these cases, the end product or even the intermediates can be separated into enantiomeric compounds by chemical, biochemical or physical measures known to the person skilled in the art or even employed as such in the synthesis.

After removal of the solvent, the compounds of the formula I can be obtained by conventional work-up steps, such as, for example, addition of water to the reaction mixture and extraction. It may be advantageous sub-sequentially to carry out a distillation or crystallisation for further purification of the product.

The invention furthermore relates to medicaments comprising at least one compound according to the invention and/or physiologically acceptable salts, derivatives, solvates and stereoisomers thereof, including mixtures thereof in all ratios.

A pharmaceutical composition according to the invention may furthermore comprise further excipients and/or adjuvants and optionally one or more further medicament active ingredients.

The invention furthermore relates to a process for the preparation of a medicament, characterised in that a compound according to the invention and/or one of its physiologically acceptable salts, derivatives, solvates and stereoisomers, including mixtures thereof in all ratios, is brought into a suitable dosage form together with a solid, liquid or semi-liquid excipient or adjuvant.

The invention also relates to a set (kit) consisting of separate packs of
a) an effective amount of a compound according to the invention and/or physiologically acceptable salts, derivatives, solvates and stereoisomers thereof, including mixtures thereof in all ratios, and
b) an effective amount of a further medicament active ingredient.

The set comprises suitable containers, such as boxes, individual bottles, bags or ampoules. The set may, for example, comprise separate ampoules, each containing an effective amount of a compound according to the invention and/or pharmaceutically usable derivatives, solvates and stereoisomers thereof, including mixtures thereof in all ratios, and an effective amount of a further medicament active ingredient in dissolved or lyophilised form.

Medicaments can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, sex, weight and condition of the patient. Preferred dosage unit formulations are those which comprise a daily dose or part-dose, as indicated above, or a corresponding fraction thereof of an active ingredient. Furthermore, medicaments of this type can be prepared using a process which is generally known in the pharmaceutical art.

Medicaments can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such medicaments can be prepared using all processes known in the pharmaceutical art by, for example, combining the active ingredient with the excipient(s) or adjuvant(s).

Medicaments adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units. Oral liquids, such as, for example, solution, syrups and elixirs, can be prepared in the form of dosage units so that a given quantity comprises a prespecified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil, or natural sweeteners or saccharin or other artificial sweeteners, and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Medicaments adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(4318, 1986.

Medicaments adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Medicaments adapted for topical application to the eye include eye drops, in which the active ingredient is dissolved or suspended in a suitable carrier, in particular an aqueous solvent.

Medicaments adapted for topical application in the mouth encompass lozenges, pastilles and mouthwashes.

Medicaments adapted for rectal administration can be administered in the form of suppositories or enemas.

Medicaments adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Medicaments adapted for administration by inhalation encompass finely particulate dusts or mists, which can be generated by various types of pressurised dispensers with aerosols, nebulisers or insufflators.

Medicaments adapted for vaginal administration can be administered as pessaries, tampons, creams, gels, pastes, foams or spray formulations.

Medicaments adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multi-dose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the medicaments according to the invention may also comprise other agents usual in the art with respect to the particular type of pharmaceutical formulation; thus, for example, medicaments which are suitable for oral administration may comprise flavours.

A therapeutically effective amount of a compound of the present invention depends on a number of factors, including, for example, the age and weight of the recipient, the precise condition that requires treatment, and its severity, the nature of the formulation and the method of administration, and is ultimately determined by the treating doctor or vet. However, an effective amount of a compound of the formula I for the treatment of the diseases according to the invention is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or more usually in a series of part-doses (such as, for example, two, three, four, five or six) per day, so that the total daily dose is the same. An effective amount of a salt or solvate or of a physiologically functional derivative thereof can be determined as a fraction of the effective amount of the compound according to the invention per se.

The compounds according to the invention exhibit an advantageous biological activity which can easily be detected in enzyme assays. In such enzyme-based assays, the compounds according to the invention preferably exhibit and cause an inhibiting effect, which is usually documented by 1050 values in a suitable range, preferably in the micromolar range and more preferably in the nanomolar range.

The present invention relates to compounds according to the invention as effectors, preferably as inhibitors of the signalling pathways described here. The invention therefore particularly preferably relates to compounds according to the invention as activators and inhibitors of serine/threonine and tyrosine kinases, preferably as inhibitors of cytosolic and receptor tyrosine kinases, in particular receptor tyrosine kinases.

As discussed above, the signalling pathways influenced by the compounds according to the invention are relevant for various diseases. Accordingly, the compounds according to the invention are useful in the prophylaxis and/or treatment of diseases which are dependent on the said signalling pathways through interaction with one or more of the said signalling pathways.

The present invention therefore furthermore relates to the use of compounds according to the invention and/or physiologically acceptable salts, derivatives, solvates and stereoisomers thereof, including mixtures thereof in all ratios, for the preparation of a medicament for the treatment and/or prophylaxis of diseases, in particular diseases that are caused, mediated and/or propagated by kinases and/or by kinase-mediated signal transduction.

In addition, the present compounds are suitable as pharmaceutical active ingredients for mammals, in particular for humans, in the treatment of protein kinase-induced diseases. The expression “protein kinase-induced diseases” refers to pathological conditions which are dependent on the activity of one or more protein kinases. The kinases participate either directly or indirectly in the signal transduction pathways of a variety of cellular activities, including proliferation, adhesion and migration, as well as differentiation. Diseases associated with tyrosine kinase activity include cancer, tumour growth, arteriosclerosis, diabetic retinopathy and inflammatory diseases.

The diseases discussed here are usually divided into two groups, hyperproliferative and non-hyperproliferative diseases. In this connection, psoriasis, arthritis, inflammation, endometriosis, scarring, benign prostatic hyperplasia, immunological diseases, autoimmune diseases and immunodeficiency diseases are regarded as non-cancerous diseases, of which arthritis, inflammation, immunological diseases, autoimmune diseases and immunodeficiency diseases are usually regarded as non-hyperproliferative diseases.

In this connection, brain cancer, lung cancer, squamous cell cancer, bladder cancer, stomach cancer, pancreatic cancer, liver cancer, kidney cancer, intestinal cancer, breast cancer, head cancer, neck cancer, oesophageal cancer, gynaecological cancer, thyroid cancer, lymphomas, chronic leukaemia and acute leukaemia are to be regarded as cancerous diseases, all of which are usually counted in the group of hyperproliferative diseases. Especially cancerous cell growth and especially cancerous cell growth mediated directly or indirectly by IGF-1R is a disease which is a target of the present invention.

The present invention therefore relates to the use of compounds according to the invention for the preparation of a medicament for the treatment and/or prophylaxis of the said diseases and also to a method for the treatment of the said diseases which comprises the administration of one or more compounds according to the invention to a patient in need of such an administration.

The recipient or patient can belong to any mammalian species, for example a primate species, particularly humans; rodents, including mice, rats and hamsters; rabbits; horses, cows, dogs, cats, etc. Animal models are of interest for experimental investigations, providing a model for the treatment of human disease.

The receptiveness of a particular cell to treatment with the compounds according to the invention can be determined by in-vitro tests. Typically, a culture of the cell is incubated with a compound according to the invention at various concentrations for a period of time which is sufficient to allow the active ingredients to induce cell death or to inhibit migration, usually between about one hour and one week. In-vitro tests can be carried out using cultivated cells from a biopsy sample. The viable cells remaining after the treatment are then counted.

The dose varies depending on the specific compound used, the specific disease, the patient status, etc. A therapeutic dose is typically sufficient considerably to reduce the undesired cell population in the target tissue, while the viability of the patient is maintained. The treatment is generally continued until a considerable reduction has occurred, for example an at least about 50% reduction in the specific cell count, and may be continued until essentially no more undesired cells are detected in the body.

For the identification of protein kinase inhibitors, various assay systems are available. In scintillation proximity assay (Sorg et al., J, of. Biomolecular Screening: 7:11-19, 2002) and flashplate assay, the radioactive phosphorylation of a protein or peptide as substrate with γATP is measured. In the presence of an inhibitory compound, a decreased radioactive signal, or none at all, is detectable. Furthermore, homogeneous time-resolved fluorescence resonance energy transfer (HTR-FRET) and fluorescence polarisation (FP) technologies are suitable as assay methods (Sills et al., J. of Biomolecular Screening, 7: 191-214, 2002).

Other non-radioactive ELISA assay methods use specific phospho-anti-bodies (phospho-ABs). The phospho-AB binds only the phosphorylated substrate. This binding can be detected by chemiluminescence using a second peroxidase-conjugated anti-sheep antibody (Ross et al., Biochem. J. 366:977-981, 2002).

Assay systems with which the inhibitory action of the compounds according to the invention can be tested, especially on the protein kinases TIE2, VEGF-2, SGK1, TGFβR1, IGF1R, PDGFRβ and FAK, are described in Examples B1 to B7.

There are many diseases and conditions associated with deregulation of cell proliferation and cell death (apoptosis). The diseases and conditions that can be treated, prevented or ameliorated by compounds according to the invention include, but are not limited to, the diseases and conditions listed below. The compounds according to the invention are suitable in the treatment and/or prophylaxis of a number of different diseases and conditions where there is proliferation and/or migration of smooth muscle cells and/or inflammatory cells into the intimal layer of a vessel, resulting in restricted blood flow through that vessel, for example in the case of neointimal occlusive lesions. Occlusive transplant vascular diseases of interest include atherosclerosis, coronary vascular disease after transplantation, vein graft stenosis, peri-anastomotic prosthetic restenosis, restenosis after angioplasty or stent placement and the like.

The present invention encompasses the use of the compounds according to the invention for the treatment or prevention of cancer. In particular, the invention relates to the use of compounds according to the invention and/or physiologically acceptable salts, derivatives, solvates and stereoisomers thereof, including mixtures thereof in all ratios, for the preparation of a medicament for the treatment and/or prophylaxis of solid tumours, where the solid tumour is particularly preferably selected from the group consisting of brain tumour, tumour of the urogenital tract, tumour of the lymphatic system, stomach tumour, laryngeal tumour, lung tumour. Solid tumours selected from the group consisting of monocytic leukaemia, lung adenocarcinoma, small-cell and non-small-cell lung carcinomas, renal cell carcinoma, endometrial carcinoma, multiple myeloma, prostate cancer, colorectal cancer, pancreatic cancer, glioblastomas and breast carcinoma can preferably also be treated with medicaments comprising compounds according to the invention.

The compounds according to the invention can be administered to patients for the treatment of cancer. The present compounds inhibit tumour angiogenesis via binding to protein kinases and thus influence the growth of tumours. The properties of the compounds according to the invention may also appear suitable for the treatment of certain forms of blindness which are associated with retinal neovascularisation.

The invention therefore also relates to the use of compounds according to the invention and/or physiologically acceptable salts, derivatives, solvates and stereoisomers thereof, including mixtures thereof in all ratios, for the preparation of a medicament for the treatment and or prophylaxis of diseases which are caused, mediated and/or propagated by angiogenesis.

A disease of this type in which angiogenesis is involved is an ocular disease, such as retinal vascularisation, diabetic retinopathy, age-induced macular degeneration and the like.

The invention therefore also relates to the use of the compounds according to the invention for the preparation of a medicament for the treatment and/or prophylaxis of the above diseases.

The use of compounds according to the invention and/or physiologically acceptable salts and solvates thereof for the preparation of a medicament for the treatment and/or prophylaxis of inflammatory diseases likewise falls within the scope of the present invention. Inflammatory diseases of this type include, for example, rheumatoid arthritis, psoriasis, contact dermatitis, late-type hypersensitivity reaction and the like.

Preference is given to the use for the treatment of diseases, preferably from the group of hyperproliferative and non-hyperproliferative diseases. These are cancer diseases or non-cancerous diseases.

The invention also relates to the use of compounds according to the invention and/or physiologically acceptable salts, derivatives, solvates and stereoisomers thereof, including mixtures thereof in all ratios, for the preparation of a medicament for the treatment of diseases selected from the group of non-cancerous diseases consisting of psoriasis, arthritis, inflammation, endometriosis, scarring, benign prostatic hyperplasia, immunological diseases, autoimmune diseases and immunodeficiency diseases.

The invention furthermore relates to the use of compounds according to the invention and/or physiologically acceptable salts, derivatives, solvates and stereoisomers thereof, including mixtures thereof in all ratios, for the preparation of a medicament for the treatment of diseases selected from the group of cancerous diseases consisting of brain cancer, lung cancer, squamous epithelial cancer, bladder cancer, stomach cancer, pancreatic cancer, liver cancer, kidney cancer, colorectal cancer, breast cancer, head cancer, neck cancer, oesophageal cancer, gynaecological cancer, thyroid cancer, lymphoma, multiple myeloma, chronic leukaemia and acute leukaemia.

The present compounds are also suitable for combination with known anti-cancer agents. These known anti-cancer agents include the following oestrogen receptor modulators, androgen receptor modulators, retinoid receptor modulators, cytotoxic substances, antiproliferative agents, prenyl-protein transferase inhibitors, HMG-CoA reductase inhibitors, HIV protease inhibitors, reverse transcriptase inhibitors, growth factor inhibitors and angiogenesis inhibitors. The present compounds are particularly suitable for administration at the same time as radiotherapy.

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenylretinamide.

“cytotoxic substances” refers to compounds which result in cell death primarily through direct action on the cellular function or which inhibit or interfere with cell mitosis, including alkylating agents, tumour necrosis factors, intercalators, microtubulin inhibitors and topoisomerase inhibitors. Examples of cytotoxic substances include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)-mu-[diamineplatinum(II)]bis[diamine(chloro)platinum(II)]tetrachloride, diarizidinyispermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, vairubicin, amrubicin, antineoplaston, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)-dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexo-hydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]-acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno-[2,1-c]quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, neizarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo-(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal anti-bodies to growth factors, such as cetuximab, matuzumab, and tumour suppressor genes, such as p53, which can be delivered via recombinant virus-mediated gene transfer (see U.S. Pat. No. 6,069,134, for example).

### WORKING EXAMPLES

### Example A1

### Preparation of 4-chloro-5-iodopyrrolo[2,3-d]pyrimidine in Accordance with the Following Reaction Scheme

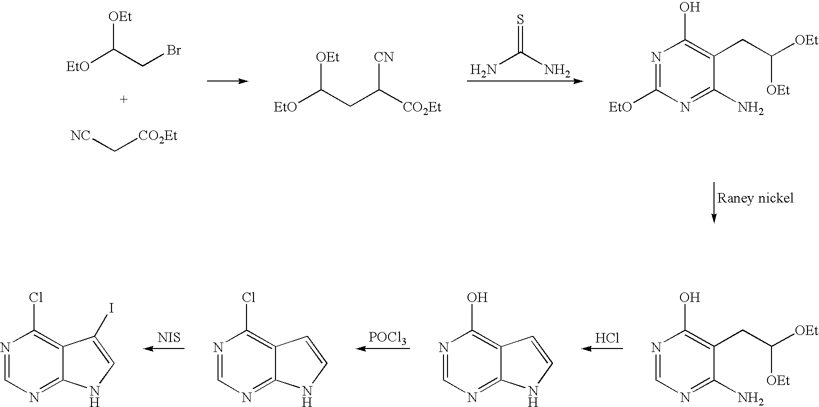

(a) 130 ml (860 mmol) of bromoacetaldehyde diethyl acetal are refluxed for 10 h with ethyl cyanoacetate (430 ml, 4.04 mol), sodium iodide (8.1 g; 54.04 mmol) and potassium carbonate (115.9 g; 839 mmol). After cooling to room temperature (RT), the batch is stirred with 800 ml of water, the aqueous phase is extracted with diethyl ether, the combined organic phases are dried and evaporated. Chromatography gives 124.99 g (63%) of a colourless liquid ethyl 2-cyano-4,4-diethoxybutyrate.

(b) 3.3 g (99 mmol) of sodium are added at 0° C. to 75 ml of ethanol, and 7.5 g (99 mmol) of thiourea and ethyl 2-cyano-4,4-diethoxybutyrate (20.6 g; 90 mmol) are added when the sodium has completely reacted. The mixture is then refluxed for 10 h, and, after cooling, the solvent is removed in vacuo. The residue is partitioned between water and ether, and, after phase separation, the aqueous phase is acidified using 5.7 ml (99 mmol) of acetic acid. The product precipitates out, is filtered off and dried in vacuo, giving 21.73 g (93%) of 6-amino-5-(2,2-diethoxyethyl)-2-mercapto-pyrimidin-4-ol in the form of a beige powder, which is employed in the subsequent reaction without further purification.

(c) 35.5 g (187 mmol) of 6-amio-5-(2,2-diethoxyethyl)-2-mercaptopyrimidin-4-ol are added to a suspension of 50 g of Raney nickel in water and 50 ml of ammonia (26% in water), and this is warmed under reflux for one hour. The mixture is filtered while hot, and the ammonia is evaporated off. 100 ml of 3 N HCl are added to the aqueous residue, and the mixture is stirred at RT for 48 hours. The product precipitating out is filtered off and washed with water and ether, giving 15.46 g (83.5%) of a colourless solid 7H-pyrrolo[2,3-d]pyrimidin-4-ol, whose melting point is above 300° C.

(d) The mixture of 5 g (37 mmol) of 7H-pyrrolo[2,3-d]pyrimidin-4-ol in 50 ml of POCl₃ is warmed until the starting material is completely in solution. The mixture is then stirred for a further 45 minutes without further supply of heat, and finally the POCl₃ is removed in vacuo. The residue is taken up in ice-water, rendered alkaline using NaHCO₃ and extracted with ether. The organic phase is dried and evaporated, giving 5.28 g (93%) of 4-chloropyrrolo[2,3-d]pyrimidine in the form of a greenish solid which melts at 187-188° C.

(e) 5 g (32.56 mmol) of 4-chloropyrrolo[2,3-d]pyrimidine and 11 g (50 mmol) of N-iodosuccinimide are dissolved in 60 ml of DMF, and the mixture is stirred at RT for 10 hours. The mixture is worked up using aqueous sodium thiosulfate solution and extracted with ether. The organic phase is washed with aqueous sodium thiosulfate solution and ammonium chloride solution. The solid which finally remains behind is recrystallised from methanol, giving 7 g (77%) of 4-chloro-5-iodopyrrolo[2,3-d]pyrimidine in the form of a pale-brownish solid which melts at 195-196° C.

### Example A2

### Preparation of (3-hydroxycyclobutylmethyl)-tent-butyl Carbamate in Accordance with the Following Reaction Scheme

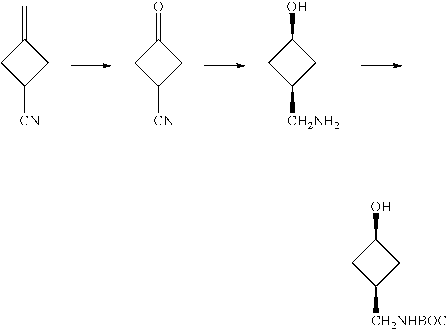

(a) 50 g (0.54 mol) of 3-methylenecyclobutanecarbonitrile are dissolved in 600 ml of water and 50 ml of ether, cooled to 5° C., and 100 mg (0.4 mmol) of osmium(IV) oxide are added. 260 g (1.2 mol) of sodium periodate are then added in portions at the temperature indicated, and the mixture is allowed to warm to RT. The organic phase is extracted with dichloromethane and, after drying and evaporation, chromatographed over silica gel, giving 25 g (49%) of 3-oxocyclobutanecarbonitrile as colourless crystals (m.p. 51° C.).

(b) 25 g (0.26 mop of 3-oxocyclobutanecarbonitrile are dissolved in 350 ml of THF and added dropwise to a suspension of 40 g (1.05 mol) of lithium aluminium hydride in 350 ml of THF. After two hours, the batch is cooled to 0° C., and water is added, the mixture is filtered, and the residue is evaporated to dryness. Chromatography over silica gel gives 20 g (70%) of 3-aminomethylcyclobutanol as colourless oil.

(This alanate reduction gives exclusively the syn product in a substrate-controlled process.)

(c) 10 g (0.1 mol) of 3-aminomethylcyclobutanol are dissolved in 200 ml of THF, and 33 g (0.15 mol) of di-tert-butyl dicarbonate and 14 ml (0.1 mol) of triethylamine are added. After stirring at RT for 10 h, the mixture is evaporated to dryness and fractionated over silica gel, giving 11 g (55%) of (3-hydroxycyclobutylmethyl)-tert-butyl carbamate in the form of colourless crystals which melt at 109-110° C.

### Example A3

### Inversion of the Absolute Configuration at the Carbinol Centre by the Mitsunobu Method in Accordance with the Following Reaction Scheme (Optional)

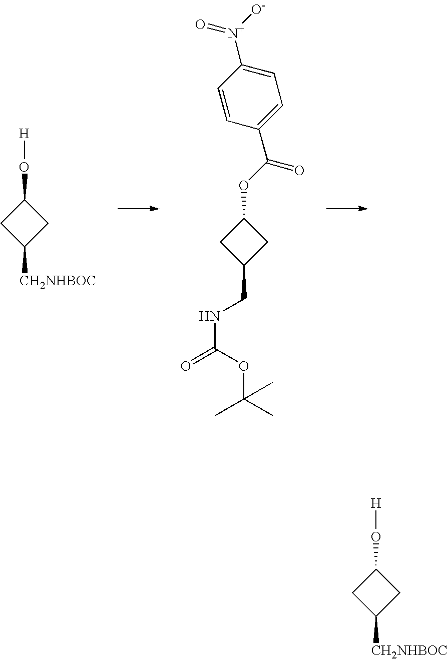

(a) (3-Hydroxycyclobutylmethyl)-tert-butyl cis-carbamate is dissolved in THF and cooled to 0° C. in the presence of triphenylphosphine and 4-nitrobenzoic acid. Diisopropyl azodicarboxylate is added dropwise at this temperature, and the mixture is allowed to warm to RT. After 12 h, the batch is worked up using 5% sodium hydrogencarbonate solution. Phase separation and chromatography gives 3-(tert-butoxycarbonylaminomethyl)cyclobutyl 4-nitrobenzoate.

(b) The ester is dissolved in methanol and stirred at RT for 12 h in the presence of 1 N sodium hydroxide solution. The alcohol is removed in vacuo, and the aqueous residue is extracted to exhaustion with dichloromethane. The organic phase is dried, concentrated, and the residue is chromatographed over silica gel, giving (3-hydroxycyclobutylmethyl)-tert-butyl trans-carbamate.

### Example A4

### Preparation of 7-(-3-aminomethylcyclobutyl)-5-(3-fluorophenyl)-7H-pyrrolo[2,3-d]pyrimidin-4-ylamine in Accordance with the Following Reaction Scheme

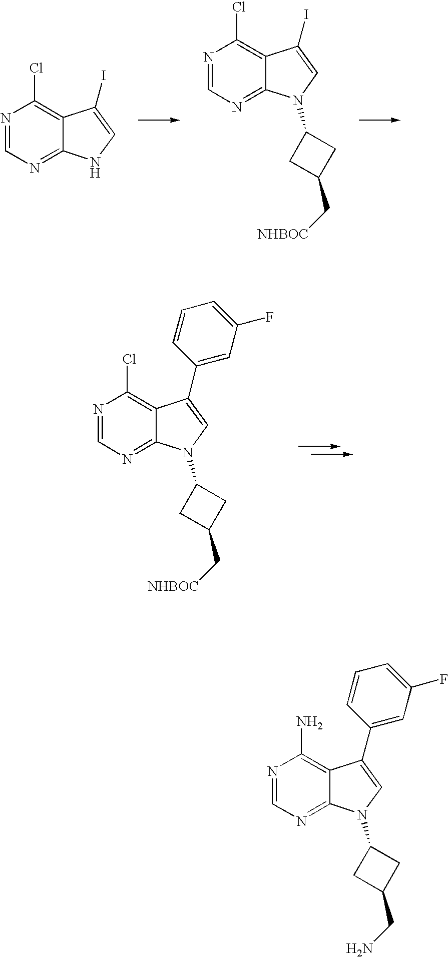

(a) 1.8 g (5.4 mmol) of triphenylphosphine in 60 ml of THF are initially introduced and cooled to −65° C. 1.1 ml (5.6 mmol) of diisopropyl azodicarboxylate are then added dropwise. After 10 minutes, the 4-chloro-5-iodopyrrolo[2,3-d]pyrimidine is added, and, after a further 15 min, the 3-hydroxycyclobutylmethyl tert-butyl cis-carbamate dissolved in 5 ml of THF is added. The batch is subsequently stirred at 50° C. for a further 10 h. After the solvent has been removed by distillation, the product is chromatographed on silica gel, giving 1.5 g of a pale oil.

This reaction procedure results in inversion at the carbinol centre, so that the radicals on the four-membered ring are in the trans-position to one another. The stereochemistry can be determined via NOE-NMR measurements.

(b) 600 mg (1.3 mmol) of [3-(4-chloro-5-iodopyrrolo[2,3-d]pyrimidin-7-yl)-cyclobutylmethyl]tert-butyl carbamate and 370 mg (1.7 mmol) of 2-(3-fluorophenyl)-4,4,5,5-tetramethyl-1,3,2-dioxaborolane are initially introduced with 450 mg of sodium carbonate (5 mmol) in 15 ml of DME and 10 ml of water. 31 mg (0.03 mmol) of tetrakis(triphenylphosphine)palladium are added to this suspension, and the mixture is warmed under reflux for 10 h. The product {3-[4-chloro-5-(3-fluorophenyl)pyrrolo[2,3-d]pyrimidin-7-yl]cyclobutylmethyl}tert-butyl carbamate is obtained by chromatography on silica gel.

(c) 500 mg (1.2 mmol) of {3-[4-chloro-5-(3-fluorophenyl)pyrrolo[2,3-d]-pyrimidin-7-yl]cyclobutylmethyl}tert-butyl carbamate are dissolved in 30 ml of ammonia water (32%) and 10 ml of THF and heated at 100° C. for 10 h in a sealed vessel together with 42 mg (0.2 mmol) of copper sulfate. The batch is neutralised and extracted with ethyl acetate. Chromatography on silica gel gives 130 mg (36%) of 7-(3-aminomethylcyclobutyl)-5-(3-fluorophenyl)-7H-pyrrolo[2,3-d]pyrimidin-4-ylamine as colourless solid. M.p.: 101-102° C.

### Example A5

### Preparation of 5-(3-fluorophenyl)-7-(3-pyrrolidin-1-ylmethylcyclobutyl)-7H-pyrrolo[2,3-d]pyrimidin-4-ylamine in Accordance with the Following Reaction Scheme

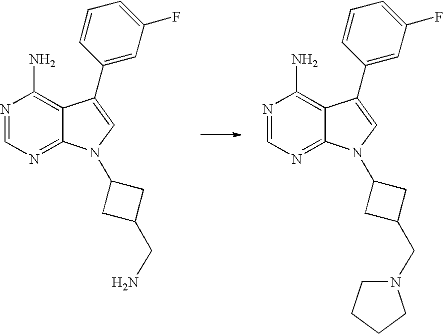

250 mg (mmol) of 7-(3-aminomethylcyclobutyl)-5-(3-fluorophenyl)-7H-pyrrolo[2,3-d]pyrimidin-4-ylamine are dissolved in 10 ml of dioxane and stirred at 80° C. for 10 h in the presence of triethylamine with 1,4-dibromobutane, giving 5-(3-fluorophenyl)-7-(3-pyrrolidin-1-ylmethylcyclobutyl)-7H-pyrrolo[2,3-d]pyrimidin-4-ylamine in a yield of 56%.

### Example A6

### Preparation of 3-[4-amino-7-(3-pyrrolidin-1-ylmethylcyclobutyl)-7H-pyrrolo[2,3-d]pyrimidin-5-yl]phenol in Accordance with the Following Reaction Scheme

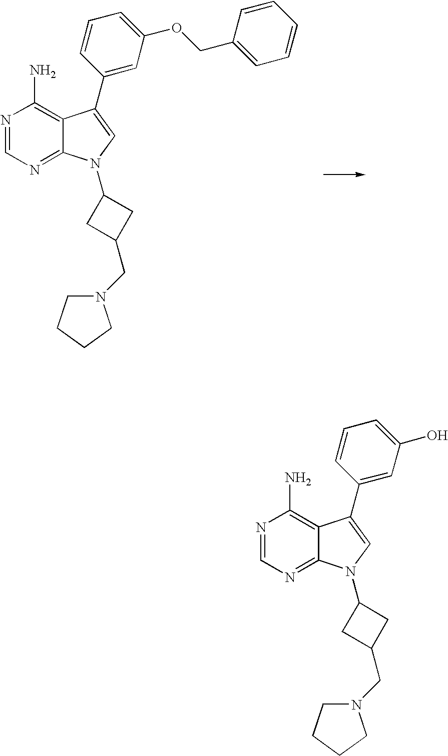

(a) Firstly, 5-(3-benzyloxyphenyl)-7-(3-pyrrolidin-1-ylmethylcyclobutyl)-7H-pyrrolo[2,3-d]pyrimidin-4-ylamine is prepared analogously to Example A3 (b) and (c) from 2-(3-benzyloxyphenyl)-4,4,5,5-tetramethyl-1,3,2-dioxaborolane and [3-(4-chloro-5-iodopyrrolo[2,3-d]pyrimidin-7-yl)cyclobutylmethyl]tert-butyl carbamate.

(b) 500 mg of this compound are dissolved in 10 ml of methanol and reacted with 25 ml of hydrogen on 100 mg of palladium on carbon, giving 250 mg (86%) of 3-[4-amino-7-(3-pyrrolidin-1-ylmethylcyclobutyl)-7H-pyrrolo[2,3-d]pyrimidin-5-yl]phenol as a colourless solid (m.p. 193-194° C.).

3-[4-Amino-7-(3-pyrrolidin-1-ylmethylcyclobutyl)-7H-pyrrolo[2,3-d]pyrimidin-5-yl]phenol has a pronounced spectrum activity (IC₅₀ in μM), which gives the compound great therapeutic importance. The inhibition constants can be determined in accordance with Examples B1 to B8 indicated below:

| IC₅₀ | Kinase |
|---|---|
| 0.06 | IGF |
| 1.8 | FAK |
| 0.52 | TIE |
| 0.18 | VEGF |
| 0.097 | PDGF |
| 11.6 | SGK |
| 0.12 | TGF |

The solubility of the compound is determined as described in Example C. In phosphate buffer (37° C., pH 7.0), it is 1071 μg/ml.

### Example B

### Inhibition of Various Protein Kinases (IC₅₀)

### B1a: Inhibition of IGF1R (Flashplate Assay)

The test plates used are 96-well flashplate microtitre plates from Perkin Elmer (USA). The components of the kinase reaction are pipetted into the assay plate. The IGF1R kinase is incubated for 1 hr. with radioactively labelled ³³P-ATP in the presence and absence of test substances in a total volume of 100 μl at room temperature together with biotinylated poly(Glu, Tyr)4:1. The reaction is terminated using 25 μl of a 200 mM EDTA solution. After incubation for a further 30 min at room temperature, the supernatants are removed by suction, and the wells are washed three times with 100 μl of 0.9% NaCl solution each time. The radioactivity is measured using a Topcount scintillation counter (PerkinElmer, USA). IC₅₀ values are calculated using the RS1 computer program.

### B1b: Inhibition of IGF1R (ELISA Assay)

Cultivated human tumour cells which express the IGF1 receptor (IGF1R) (for example MCF-7 or Calu-6) are stimulated using human IGF1, the natural ligand of IGF1R. The stimulation induces an autophosphorylation of tyrosine residues in the cytoplasmatic IGF1R domain, which triggers signal transduction cascades, which result in inhibition of apoptosis and proliferation of the cells.

The amount of phosphorylated IGF1R is determined by a receptor-specific capture ELISA or an analogous LUMINEX assay. The IGF1R from cell lysates is bound to a 96-well ELISA plate or LUMINEX beads by means of a specific antibody (“capturing”), and the tyrosine phosphorylation is detected using a biotin-labelled anti-phosphotyrosine antibody and a streptavidin-peroxidase conjugate by a chemiluminescence method or by means of a fluorescence-labelled anti-phosphotyrosine antibody.

For determination of the activity of kinase inhibitors, cells are pretreated with increasing concentrations of these compounds for 45 min and subsequently stimulated using IGF1 for 5 min. As internal control, the biological activity of the ligand IGF1 is checked, and a concentration series of an IGF1R reference inhibitor is measured.

### B2a: Inhibition of FAK (Flashplate Assay)

The test plates used are 384-well flashplate microtitre plates from Perkin Elmer (USA). The components of the kinase reaction are pipetted into the assay plate. The FAK kinase is incubated for 3 hr. with radioactively labelled ³³P-ATP in the presence and absence of test substances in a total volume of 100 μl at room temperature together with biotinylated poly(Glu, Tyr)4:1. The reaction is terminated using 25 μl of a 200 mM EDTA solution. After incubation for a further 30 min at room temperature, the supernatants are removed by suction, and the wells are washed three times with 100 μl of 0.9% NaCl solution each time. The radioactivity is measured using a Topcount scintillation counter (PerkinElmer, USA). IC₅₀ values are calculated using the RS1 computer program.

### B2b: Inhibition of FAK (ELISA Assay)

Cultivated human cells which have amplification of the FAK (focal adhesion kinase) gene (for example Calu-6 or HT-29) have constitutive FAK activation which is associated with increased tyrosine autophosphorylation. Activated FAK promotes the invasive growth of tumour cells and inhibits anoikis.

The amount of phosphorylated FAK is determined by a specific capture ELISA or an analogous LUMINEX assay. FAK from cell lysates is bound to a 96-well ELISA plate or LUMINEX beads by means of a specific antibody (“capturing”), and the tyrosine phosphorylation is detected using a biotin-labelled anti-phosphotyrosine antibody and a streptavidin-peroxidase conjugate by a chemiluminescence method or by means of a fluorescence-labelled anti-phosphotyrosine antibody.

For determination of the activity of kinase inhibitors, cells are pretreated with increasing concentrations of these compounds for 45 min, and the FAK phosphorylation is then determined. As internal control, a concentration series of a reference inhibitor is measured.

### B3: Inhibition of VEGF-2 (Flashplate Assay)

The test plates used are 384-well flashplate microtitre plates from Perkin Elmer (USA). The components of the kinase reaction are pipetted into the assay plate. The VEGF-2 kinase is incubated for 3 hr. with radioactively labelled ³³P-ATP in the presence and absence of test substances in a total volume of 100 μl at room temperature together with biotinylated poly(Glu, Tyr)4:1. The reaction is terminated using 25 μl of a 200 mM EDTA solution. After incubation for a further 30 min at room temperature, the supernatants are removed by suction, and the wells are washed three times with 100 μl of 0.9% NaCl solution each time. The radioactivity is measured using a Topcount scintillation counter (PerkinElmer, USA). IC₅₀ values are calculated using the RS1 computer program.

B4: inhibition of T1E2 (Flashplate Assay)

The test plates used are 384-well flashplate microtitre plates from Perkin Elmer (USA). The components of the kinase reaction are pipetted into the assay plate. The TIE2 kinase is incubated for 3 hr. with radioactively labelled ³³P-ATP in the presence and absence of test substances in a total volume of 100 μl at room temperature together with biotinylated poly(Glu, Tyr)4:1. The reaction is terminated using 25 μl of a 200 mM EDTA solution. After incubation for a further 30 min at room temperature, the supernatants are removed by suction, and the wells are washed three times with 100 μl of 0.9% NaCl solution each time. The radioactivity is measured using a Topcount scintillation counter (PerkinElmer, USA). IC₅₀ values are calculated using the RS1 computer program.

### B5: Inhibition of SGK1 (Flashplate Assay)

The test plates used are 384-well flashplate microtitre plates from Perkin Elmer (USA). The components of the kinase reaction are pipetted into the assay plate. The SGK1 kinase is incubated for 3 hr. with radioactively labelled ³³P-ATP in the presence and absence of test substances in a total volume of 100 μl at room temperature together with biotinylated poly(Glu, Tyr)4:1. The reaction is terminated using 25 μl of a 200 mM EDTA solution. After incubation for a further 30 min at room temperature, the supernatants are removed by suction, and the wells are washed three times with 100 μl of 0.9% NaCl solution each time. The radioactivity is measured using a Topcount scintillation counter (PerkinElmer, USA). IC₅₀ values are calculated using the RS1 computer program.

### B6: Inhibition of PDGFRβ (Flashplate Assay)

The test plates used are 96-well flashplate microtitre plates from Perkin Elmer (USA). The components of the kinase reaction are pipetted into the assay plate. The PDGFRβ kinase is incubated for 3 hr. with radioactively labelled ³³P-ATP in the presence and absence of test substances in a total volume of 100 μl at room temperature (here autophosphorylation of the kinase). The reaction is terminated using 150 μl of a 60 mM EDTA solution. After incubation for a further 30 min at room temperature, the supernatants are removed by suction, and the wells are washed three times with 200 μl of 0.9% NaCl solution each time. The radioactivity is measured using a Topcount scintillation counter (PerkinElmer, USA). IC₅₀ values are calculated using the RS1 computer program.

### B7: Inhibition of TGFβR1 (Flashplate Assay)

The test is carried out as described in Example B1b for IGF1R. A biotinylated is added to the TGFβR1 kinase.

The kinase assay is carried out as a 384-well flashplate assay.

The components of the kinase reaction are pipetted into the assay plate. The TGFβR1 kinase is incubated for 45 min with radioactively labelled ³³P-ATP in the presence and absence of test substances together with biotinylated substrate in a total volume of 35 μl. The reaction is terminated using 25 μl of 200 mM EDTA solution, removed by suction after 30 min at room temperature, and the wells are washed with three times with 100 μl of 0.9% NaCl solution each time. The radioactivity is measured using a Topcount scintillation counter (PerkinElmer, USA). IC₅₀ values are calculated using the RS1 computer program.

### B8: Inhibition of InsR (ELISA Assay)

Cultivated human cells which express the insulin receptor (InsR) (for example HepG2) are stimulated using human insulin, the natural ligand of InsR. The stimulation induces autophosphorylation of tyrosine residues in the cytoplasmatic InsR domain, which triggers signal transduction cascades, which initiate various biological reactions of the cells. The amount of phosphorylated InsR is determined by a receptor-specific capture ELISA or an analogous LUMINEX assay. The InsR from cell lysates is bound to a 96-well ELISA plate or LUMINEX beads by means of a specific antibody (“capturing”), and the tyrosine phosphorylation is detected using a biotin-labelled anti-phosphotyrosine antibody and a streptavidin-peroxidase conjugate by a chemiluminescence method or by means of a fluorescence-labelled anti-phosphotyrosine antibody. For determination of the activity of kinase inhibitors, cells are pretreated with increasing concentrations of these compounds for 45 min and subsequently stimulated using insulin for 5 min. As internal control, the biological activity of the ligand insulin is checked, and a concentration series of a reference inhibitor is measured.

Further physicochemical data and inhibition constants of compounds according to the invention are shown in Table 1.

### Example C

### Determination of the Solubility in Phosphate Buffer by the Shake Flask Method

The procedure described in Glomme et al. (J. Pharm. Sci. 94(1): 1-16, 2005) is followed. The concentration determination is carried out by HPLC with UV detection against a standard solution.

Buffer: 3.954 g of sodium dihydrogenphosphate monohydrate 6.024 g of sodium chloride +950 ml of ultrapure water. Setting of pH 7.0 using 0.1 M NaOH or 0.1 M HCl.

### Procedure:

The samples were shaken at 37° C. and 450 rpm for 24 h. After about 7 h, the pH of the samples was checked and adjusted if necessary. It was checked whether the sample was still present in excess. Just before the end of the 24 h shaking time, the samples were again checked for pH and a precipitate.

### Equipment Used:

Ultrapure water plant: MilliQ gradient, Millipore, unit: F3PN37462D
Shaker: TiMix control, Bühler
Incubation hood: TH 15, Bühler
pH meter: 766 Calimatic Knick instrument: pH 1
pH electrode: InLab 423 Mettler
HPLC: column: LiChroCart 125-4 LiChrospher 100 RP-18
   flow rate: 1.000 ml/min
   eluents:
      eluent A: 2 ml of diethylamine, for synthesis+1000 ml of methanol, LiChrosolv
      eluent B: 5 g of ammonium acetate, for analysis+5 ml of methanol, LiChrosolv+995 ml of ultrapure water

### Example C1

### Solubilities of Compounds According to the Invention

The following solubilities are obtained by the method described above:

| | Solubility |
|---|---|
| Compound | [μg/ml] |
| 7-(3-Aminomethylcyclobutyl)-5-(3-butoxyphenyl)-7H- | 1122 |
| pyrrolo[2,3-d]pyrimidin-4-ylamine | |
| 3-[4-Amino-7-(3-pyrrolidin-1-ylmethylcyclobutyl)-7H- | 1071 |
| pyrrolo[2,3-d]pyrimidin-5-yl]phenol | 1147 (tosylate) |
| 7-(3-Aminomethylcyclobutyl)-5-(1H-indol-5-yl)-7H- | 1326 |
| pyrrolo[2,3-d]pyrimidin-4-ylamine | |
| 7-(3-Aminomethylcyclobutyl)-5-(4-aminophenyl)-7H- | 1090 |
| pyrrolo[2,3-d]pyrimidin-4-ylamine | |
| 7-(3-Aminomethylcyclobutyl)-5-(3-methanesulfonyl- | >1000 |
| phenyl)-7H-pyrrolo[2,3-d]pyrimidin-4-ylamine | |

### Example C2

### Solubilities of Structurally Similar Compounds from the Prior Art (Comparative Example)

The following solubilities are obtained by the method described above:

| | Solubility |
|---|---|
| Compound from WO 02/092599 | [μg/ml] |
| 5-(3-Benzyloxyphenyl)-7-(3-pyrrolidin-1-ylmethyl- | 53 (tosylate) |
| cyclobutyl)-7H-pyrrolo[2,3-d]pyrimidin-4-ylamine | |
| N-{3-[4-Amino-5-(3-benzyloxyphenyl)pyrrolo[2,3- | 5 |
| d]pyrimidin-7-yl]cyclobutylmethyl}acetamide | |
| Piperidine-1-carboxylic acid {3-[4-amino-5-(3- | <1 |
| benzyloxy-phenyl)pyrrolo[2,3-d]pyrimidin-7-yl]cyclo- | |
| butylmethyl}amide | |
| 7-(3-Aminomethylcyclobutyl)-5-(3-benzyloxyphenyl)- | 233 |
| 7H-pyrrolo[2,3-d]pyrimidin-4-ylamine | |
| 2-{3-[4-Amino-5-(3-benzyloxyphenyl)pyrrolo[2,3- | <1 |
| d]pyrimidin-7-yl]cyclobutylmethyl}isoindole-1,3-dione | |

It becomes clear that the solubility of the compounds according to the invention exceeds that of the prior-art compounds by 5 to 1000 times.

The following examples relate to pharmaceutical compositions:

### Example D1

### Injection Vials

A solution of 100 g of an active ingredient according to the invention and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### Example D2

### Suppositories

A mixture of 20 g of an active ingredient according to the invention with 100 g of soya lecithin and 1400 g of cocoa butter is melted, poured into moulds and allowed to cool. Each suppository contains 20 mg of active ingredient.

### Example D3

### Solution

A solution is prepared from 1 g of an active ingredient according to the invention, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D4

### Ointment

500 mg of an active ingredient according to the invention are mixed with 99.5 g of Vaseline under aseptic conditions.

### Example D5

### Tablets

A mixture of 1 kg of active ingredient, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example D6

### Dragees

Tablets are pressed analogously to Example 5e and subsequently coated in a conventional manner with a coating of sucrose, potato starch, talc, tragacanth and dye.

### Example D7

### Capsules

2 kg of active ingredient are introduced into hard gelatine capsules in a conventional manner in such a way that each capsule contains 20 mg of the active ingredient.

### Example D8

### Ampoules

A solution of 1 kg of an active ingredient according to the invention in 60 l of bidistilled water is sterile filtered, transferred into ampoules, lyophilised under sterile conditions and sealed under sterile conditions. Each ampoule contains 10 mg of active ingredient.

1. Compounds of the formula I 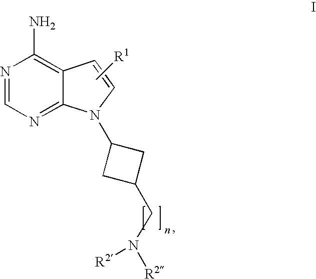
in which
R^{1′} denotes H, Hal, OH, CN, NO₂, NH₂, A, Ar,
R^{2′}, R^{2′} each, independently of one another, denote H, A having 1, 2, 3, 4, 5 or 6 C atoms, where R^{2′} and R²″, together with the N atom to which they are linked, may form a saturated or unsaturated monocyclic heterocycle having no or one further N, O or S atom,
A denotes unbranched, branched or cyclic alkyl having 1, 2, 3, 4, 5, or 6 C atoms, in which one or two CH groups may be replaced by N, furthermore in which one or two CH₂ groups may be replaced by an O, N or S atom and/or by an NH, NA, CONH, Si(CH₃)₂, NHCO, SO₂, —CH═CH— or —C≡C— group and/or, in addition, 1-7H atoms may be replaced by Hal, and in which one or two CH₃ groups may be replaced by NH, NH₂, NAH, NA₂, NHCOOA, NHCONHA, Si(CH₃)₃, CN or Ar,
Ar denotes a mono- or bicyclic aromatic homo- or heterocycle having 1 to 4 N, O and/or S atoms and 5 to 12 skeleton atoms, which may be unsubstituted or mono-, di- or trisubstituted by carbonyl oxygen, Hal, A, OH, OA, NH₂, NHA, NA₂, NO₂, CN, OCN, SCN, COOH, COOA, 2 MERCK-3522 CONH₂, CONHA, CONA₂, NHCOA, NHCOOA, NHCONH₂, NHSO₂A, CHO, COA, SO₂CH₃ and/or SO₂NH₂,
Hal denotes F, Cl, Br or I and
n denotes 0, 1, 2 or 3,
and pharmaceutically acceptable salts, derivatives, solvates and stereoisomers thereof, including mixtures thereof in all ratios.

2. Compounds according to claim 1 which conform to the formula II 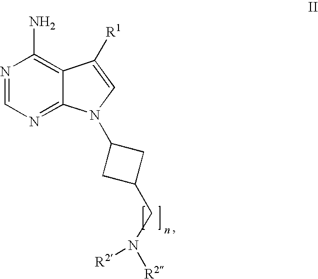
in which R¹, R² and n have the meaning indicated for the formula I and pharmaceutically acceptable salts, derivatives, solvates and stereoisomers thereof, including mixtures thereof in all ratios.

3. Compounds according to claim 1, in which the radicals not designated in greater detail have the meaning indicated for the formula I according to claim 1, but in which
in sub-formula A
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
and R² and n have the meaning indicated for the formula I,
in sub-formula B
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl, R², R²″ are H or together form a butylene unit and
n denotes 1,
in sub-formula C
R¹ denotes A, or phenyl, indolyl, indazolyl, benzotriazolyl, benzoxazolyl-2-one, furyl, thienyl, thiazolyl, pyridyl or pyrimidinyl, each of which is unsubstituted or mono- or disubstituted by A, fluorine, chlorine, bromine, iodine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, benzyloxy, methanesulfonyl, sulfonamido, methylsulfonamido, ethylsulfonamido, propylsulfonamido, butylsulfonamido, dimethylsulfonamido, phenylsulfonamido, carboxyl, methoxycarbonyl, ethoxycarbonyl or aminocarbonyl,
R^{2′}, R^{2″} are each, independently of one another, H or unbranched or branched alkyl having 1, 2, 3 or 4 C atoms, where R² and R²″ together form an ethylene, propylene, butylene or pentylene unit and
n denotes 1,
in sub-formula D
R¹ denotes propan-1-olyl, propen-1-olyl, propyn-1-olyl, or phenyl, indolyl, indazolyl, benzofuranyl, benzotriazolyl, benzimidazolyl-2-one, benzoxazolyl-2-one, furyl, thienyl, thiazolyl, pyridyl or pyrimidinyl, each of which is unsubstituted or mono- or disubstituted by hydroxyl, amino, fluorine, butoxy, acetamido, t-butoxycarbonylamino, nitro, benzyl, (dimethylphenylsilanyl)methoxy, dimethylphenylsilanyloxy, methanesulfonyl, sulfonamido, methanesulfonamido, methyl, 2-propyl, trifluoromethyl, trifluoromethoxy, trifluoromethanesulfonic acid, benzylamino, N-benzylpropane-1,3-diamino
and R² and n have the meaning indicated for the formula I,
in sub-formula E
R¹ denotes propan-1-olyl, propen-1-olyl, propyn-1-olyl, or phenyl, indolyl, indazolyl, benzofuranyl, benzotriazolyl, benzimidazolyl-2-one, benzoxazolyl-2-one, furyl, thienyl, thiazolyl, pyridyl or pyrimidinyl, each of which is unsubstituted or mono- or disubstituted by hydroxyl, amino, fluorine, butoxy, acetamido, t-butoxycarbonylamino, nitro, benzyl, (dimethylphenylsilanyl)-methoxy, dimethylphenylsilanyloxy, methanesulfonyl, sulfonamido, methanesulfonamido, methyl, 2-propyl, trifluoromethyl, trifluoromethoxy, trifluoromethanesulfonic acid, benzylamino, N-benzylpropane-1,3-diamino,
R², R²″ are H or together form a butylene unit and
n denotes 1
and pharmaceutically acceptable salts, derivatives, solvates and stereoisomers thereof, including mixtures thereof in all ratios.

4. Process for the preparation of compounds of the formula I and physiologically acceptable salts, derivatives, solvates and stereoisomers thereof, characterised in that
a compound of the formula VI 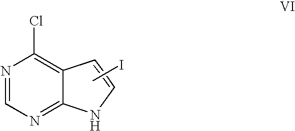
is condensed with a compound of the formula V 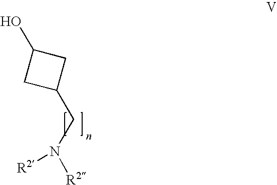
to give a compound of the formula IV 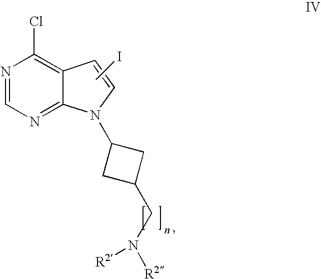
which is further linked to a desired radical R¹ to give a compound of the formula III 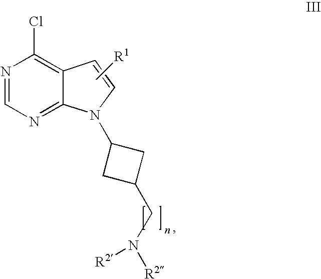
which is finally reacted with NH₃ to give a compound of the formula I, and in that, if desired, a base or acid of the formula I is converted into one of its salts.

5. Compounds according to formula I of claim 1 and/or physiologically acceptable salts, derivatives, solvates and stereoisomers thereof, including mixtures thereof in all ratios, as medicaments.

6. Medicaments comprising at least one compound according to formula I of claim 1 and/or physiologically acceptable salts, derivatives, solvates and stereoisomers thereof, including mixtures thereof in all ratios, and optionally excipients and/or adjuvants.

7. Medicaments comprising at least one compound according to formula I of claim 1 and/or physiologically acceptable salts, derivatives, solvates and stereoisomers thereof, including mixtures thereof in all ratios, and at least one further medicament active ingredient.

8. Set (kit) consisting of separate packs of
a) an effective amount of a compound according to formula I of claim 1 and/or physiologically acceptable salts, derivatives, solvates and stereoisomers thereof, including mixtures thereof in all ratios, and
b) an effective amount of a further medicament active ingredient.

9. Compounds according to formula I of claim 1 and physiologically acceptable salts, derivatives, solvates and stereoisomers thereof, including mixtures thereof in all ratios, as inhibitors of protein kinases.

10. A method of using a compounds according to claim 1 comprising preparing a medicament with a compound of claim 1 for the prophylaxis or treatment of diseases in which inhibition of protein kinases results in an improvement in the clinical picture.

11. A method of using a compound of claim 1 comprising preparing a medicament with a compound of claim 1 for the prophylaxis or treatment of cancer, tumour growth, tumour angiogenesis, arteriosclerosis, diabetic retinopathy and inflammatory diseases.

12. A method of using a compound of claim 1 comprising preparing a medicament with a compound of claim 1 for the prophylaxis or treatment of breast cancer, brain cancer, prostate cancer, colorectal cancer, small-cell lung cancer, non-small-cell lung cancer, multiple myeloma and renal-cell carcinoma and endometrial carcinoma.
